(19) United States
(12) Patent Application Publication
(10) Pub. No.: US 2023/0055143 A1
(43) Pub. Date: Feb. 23, 2023

(54) Compositions and Methods of Treating Cancer with Chimeric Antigen Receptors Targeting Glypican 3

(71) Applicant: MEDIMMUNE, LLC, GAITHERSBURG, MD (US)

(72) Inventors: Ryan GILBRETH, Gaithersburg, MD (US); Gordon MOODY, Gaithersburg, MD (US); Maria Letizia GIARDINO TORCHIA, Gaithersburg, MD (US); Qiong WANG, Gaithersburg, MD (US); John MUMM, Gaithersburg, MD (US)

(21) Appl. No.: 17/783,463

(22) PCT Filed: Dec. 11, 2020
(86) PCT No.: PCT/US2020/064415

§ 371 Date: Jun. 8, 2022

Related U.S. Application Data
(60) Provisional application No. 62/951,309, filed on Dec. 20, 2019.

(51) Int. Cl. C07K 16/30; C07K 14/725; C07K 14/705; C07K 14/715; A61K 35/17; A61K 39/395; A61P 35/00

(52) U.S. Cl. CPC C07K 16/303; C07K 14/7051; C07K 14/70521; C07K 14/70578; C07K 14/7153; A61K 35/17; A61K 39/3955; A61P 35/00; C07K 2317/622; A61K 39/00

(57) ABSTRACT

This disclosure relates to compositions and methods for treating cancer using chimeric antigen receptor T cells targeting glypican 3.

### BACKGROUND OF DISCLOSURE

### Field of Invention

This disclosure relates to treatment of cancer using chimeric antigen receptor T cells.

### Technical Background

### 1. Chimeric Antigen Receptor T Cell Therapy

Chimeric antigen receptor (CAR) T cell therapy is a specific form of cell-based immunotherapy that uses engineered T cells to fight cancer. In CAR T cell therapy, T cells are harvested from a patient's blood, engineered ex vivo to express CARs containing both antigen-binding and T cell-activating domains, expanded into a larger population, and administered to the patient. The CAR T cells act as a living drug, binding to cancer cells and bringing about their destruction. When successful, the effects of CAR T cell treatment tend to be long lasting, as evidenced by detection of CAR T cell persistence and expansion in the patients long after clinical remission.

### 2. CAR Structure and Function

The antigen-binding domain of a CAR is an extracellular region that targets a surface antigen on tumor cells. Appropriate target antigens can be proteins, phosphorylated proteins, peptide-MHC, carbohydrates, or glycolipid molecules. Ideal target antigens are widely expressed on tumor cells to enable targeting of a high percentage of the cancer cells. Ideal candidate target antigens are also usually minimally expressed on normal tissues, limiting off-tumor, on-target toxicity. The antigen-binding domain of a CAR comprises a targeting moiety, such as an antibody single chain variable fragment (scFv), which is directed against the target antigen.

The T cell-activating domain of a CAR is intracellular and activates the T cell in response to the antigen-binding domain interacting with its target antigen. A T cell activating domain can contain one or more co-stimulatory domains, which are the intracellular domains of known activating T cell receptors. The selection and positioning of costimulatory domains within a CAR construct influence CAR T cell function and fate, as costimulatory domains have differential impacts on CAR T cell kinetics, cytotoxic function, and safety profile.

The extracellular antigen-binding and intracellular T cell-activating domains of CARs are linked by a transmembrane domain, hinge, and optionally a spacer region. The hinge domain is a short peptide fragment that provides conformational freedom to facilitate binding to the target antigen on the tumor cell. It may be used alone or in conjunction with a spacer domain that projects the scFv away from the T cell surface. The optimal length of the spacer depends on the proximity of the binding epitope to the cell surface.

CAR T therapy against the B-lymphocyte antigen CD19 (Kymriah®, Novartis) has shown promise in pediatric acute lymphocytic leukemia, and CAR T therapy against B-cell maturation antigen (“bb2121,” a Celgene® and Bluebirdbio® collaboration) has shown promise against relapsed/refractory multiple myeloma. More recent data suggest that the CAR approach can be efficacious against solid tumors. A GD2 CAR natural killer T cell (NKT) therapy has shown activity in neuroblastoma (Heczey A, et al. Invariant NKT cells with chimeric antigen receptor provide a novel platform for safe and effective cancer immunotherapy. Blood;124(18):2824-33, 2014), and mesothelin CAR T with pembrolizumab has demonstrated anti-tumor activity in mesothelioma. However, additional targets for treating solid tumors are needed.

### 3. Challenges of CAR T Cell Therapy

Unfortunately, the complexities of CAR T cell-based therapy can lead to undesirable and unsafe effects. Off-tumor effects such as neurotoxicity and acute respiratory distress syndrome are potential adverse effects of CAR T cell therapy and are potentially fatal. Cytokine release syndrome (CRS) is the most common acute toxicity associated with CAR T cells. CRS occurs when lymphocytes are highly activated and release excessive amounts of inflammatory cytokines. Serum elevations of interleukin 2, interleukin 6, interleukin 1 beta, GM-CSF, and/or C-reactive protein are sometimes observed in patients with CRS when these factors are assayed. CRS is graded in severity and is diagnosed as one of grades 1-4 (mild to severe), with more serious cases clinically characterized by high fever, hypotension, hypoxia, and/or multi-organ toxicity in the patient. One study reported that 92% of acute lymphocytic leukemia patients treated with an anti-CD19 CAR T cell therapy experienced CRS, and 50% of these patients developed grade 3-4 symptoms.

Therefore, additional CAR T cell-based therapies are needed to augment the armamentarium of effective cancer treatments. However, new CAR T cell therapies must be devised that effectively treat cancer while minimizing the risk of developing dangerous inflammatory responses, such as CRS.

### BRIEF SUMMARY OF THE INVENTION

This disclosure describes compositions and methods for using CAR T cells to treat cancer.

As described below, in a first aspect, the disclosure provides an isolated nucleic acid sequence encoding a chimeric antigen receptor (CAR), wherein the CAR comprises an antigen binding domain specific for glypican 3 (GPC3), wherein the antigen binding domain has an equilibrium dissociation constant (K_{D}) of about 100 nanomolar (nM) or less, and wherein the CAR construct does not induce cytokine production in GPC3-expressing cells.

In some embodiments of the first aspect, the antigen binding domain of CAR comprises an antibody or antigen-binding fragment thereof.

In some embodiments the first aspect, the antigen binding domain is a Fab or a single chain variable fragment (scFv).

In some embodiments the first aspect, the antigen binding domain is an scFv comprising the nucleic acid sequence of SEQ ID NO: 33 or SEQ ID NO: 34.

In some embodiments of the first aspect, the isolated nucleic acid further encodes a transmembrane domain, a costimulatory domain, and a signal domain.

In some embodiments of the first aspect, wherein the transmembrane domain comprises a CD28 transmembrane domain.

In some embodiments of the first aspect, the costimulatory domain comprises one or more of CD28, 4-1BB, CD3zeta, OX-40, ICOS, CD27, GITR, and MyD88/CD40 costimulatory domains.

In some embodiments of the first aspect, the costimulatory domain comprises one or more of CD28, 4-1BB, and CD3zeta costimulatory domains.

In some embodiments of the first aspect, wherein the signal domain comprises a sequence encoding a CSFR2 signal peptide.

In some embodiments of the first aspect, the anti-GPC3 CAR further comprises a hinge/spacer domain.

In some embodiments of the first aspect, the hinge/spacer domain is an IgG4P hinge/spacer.

In some embodiments of the first aspect, the nucleic acid sequence comprises SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 26.

In a second aspect, the disclosure provides an anti-GPC3 chimeric antigen receptor (CAR) comprising an antigen binding domain, wherein the antigen binding domain comprises an antibody, Fab, or an scFv comprising a heavy chain variable region (VH) and a light chain variable region (VL), wherein the VH comprises a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39, and wherein the VL comprises a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

In some embodiments of the second aspect, the VH comprises the amino acid sequence of SEQ ID NO: 27 or SEQ ID NO: 29.

In some embodiments of the second aspect, the VL comprises the amino acid sequence of SEQ ID NO: 28 or SEQ ID NO: 30.

In some embodiments of the second aspect, the anti-GPC3 CAR further comprises a transmembrane domain, a costimulatory domain, and a signal domain.

In some embodiments of the second aspect, the anti-GPC3 CAR comprises the amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25.

In a third aspect, the disclosure provides a vector comprising a nucleic acid sequence encoding a chimeric antigen receptor (CAR), wherein the nucleic acid sequence comprises SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

In some embodiments, the disclosure provides a cell comprising the vector of the third aspect.

In a fourth aspect, the disclosure provides a cell comprising a nucleic acid sequence encoding a chimeric antigen receptor (CAR), wherein the CAR comprises an antigen binding domain specific for glypican 3 (GPC3), wherein the antigen binding domain has an equilibrium dissociation constant (K_{D}) of about 100 nanomolar (nM) or less, and wherein the CAR construct does not induce cytokine production in GPC3-cells.

In some embodiments of the fourth aspect, the nucleic acid sequence comprises SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

In a fifth aspect, the disclosure provides a cell comprising an anti-GPC3 chimeric antigen receptor (CAR) comprising an antigen binding domain, wherein the antigen binding domain comprises an antibody, Fab, or an scFv comprising a heavy chain variable region (VH) and a light chain variable region (VL), wherein the VH comprises a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39, and wherein the VL comprises a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

In some embodiments of the fifth aspect, the VH comprises the amino acid sequence of SEQ ID NO: 27 or SEQ ID NO: 29.

In some embodiments of the fifth aspect, the VL comprises the amino acid sequence of SEQ ID NO: 28 or SEQ ID NO: 30.

In some embodiments of the fifth aspect, the CAR further comprises a transmembrane domain, a costimulatory domain, and a signal domain.

In some embodiments of the fifth aspect, the CAR comprises the amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25.

In some embodiments of the fifth aspect, the cell is selected from the group consisting of a T cell, a Natural Killer (NK) cell, a cytotoxic T lymphocyte (CTL), and a regulatory T cell.

In some embodiments of the fifth aspect, the cell exhibits an anti-tumor immunity upon contacting a tumor cell expressing GPC3.

In a sixth aspect, the disclosure provides a method of treating cancer, comprising: administering to a subject in need thereof an effective amount of a cell comprising an anti-GPC3 chimeric antigen receptor (CAR) comprising an antigen binding domain, wherein the antigen binding domain comprises an antibody, Fab, or an scFv comprising a heavy chain variable region (VH) and a light chain variable region (VL), wherein the VH comprises a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39, and wherein the VL comprises a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

In some embodiments of the sixth aspect, the method further comprises inhibiting tumor growth, inducing tumor regression, and/or prolonging survival of the subject.

In some embodiments of the sixth aspect, the cell is an autologous cell.

In some embodiments of the sixth aspect, the autologous cell is selected from the group consisting of a T cell, a Natural Killer (NK) cell, a cytotoxic T lymphocyte (CTL), and a regulatory T cell.

In some embodiments of the sixth aspect, the cancer is a solid tumor.

In some embodiments of the sixth aspect, the cancer is hepatocellular carcinoma, non-small cell lung cancer, ovarian cancer, and/or squamous cell lung carcinoma.

In some embodiments of the sixth aspect, the cancer is hepatocellular carcinoma.

In some embodiments of the sixth aspect, the method further comprises administering to the subject an effective amount of an anti-TNFα antibody.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure. The drawings illustrate one or more embodiment(s) of the disclosure, and together with the description serve to explain the principles and operation of the disclosure.

FIGS. 1A and 1B. GPC3 expression in cancer and normal tissue. 1A. Anti-GPC3 antibody staining results in hepatocellular carcinoma (HCC), non-small cell lung cancer (NSCLC), and ovarian cancer. 1B. Immunohistochemistry (IHC) results for human colonic ganglion tissue.

FIG. 2. Comparison of heavy and light variable regions of single chain variable fragments (scFvs). GPC3-1 and GPC3-2 are shown.

FIG. 3A. Cell surface GPC3 CAR binding to soluble GPC3 protein. K_{D} values shown (fits shown by solid lines).

FIGS. 3B and 3C. Surface plasmon resonance binding of anti-GPC3 scFv-Fcs to soluble GPC3 protein. Mean values of kₐ, k_{d}, and K_{D} are reported for both interactions (fits shown in solid lines).

FIGS. 4A and 4B. Production of cytokines upon in vitro administration of chimeric antigen receptor (CAR) constructs to cells with and without target antigen. GPC3 CAR T confer antigen-specific cytokine production. Cell lines are shown from left to right for each construct in the order listed from top to bottom in the legend. 4A. Results for three cytokines are displayed. UT, untransduced T cells, which are donor T cells that are activated and expanded but not transduced with a CAR transgene. 4B. Results for interferon gamma (IFN-γ) are shown for a subset of the constructs. Cell types (all negative, with the exception of HEPG2) are shown in the legend at right.

FIG. 5. Cytotoxicity of CARs in HCC cell lines. Constructs used are shown in the legend at right. E:T ratio, effector: target ratio. UT, untransduced T cells.

FIG. 6. Cytotoxicity of GPC3-1 in HCC cell lines expressing low level of GPC3. 6A GPC3 expression evaluated by flow cytometry on the cell lines indicated. 6B. Receptor density on the indicated cell lines. 6C. cytotoxicity of GPC3-1 against cell lines indicated in the legend, at 3:1 (upper graph) and 0.3:1 (lower graph) Effector:Target ratio. 6D. KT50 (time to kill 50% of the target) of GPC3-1 against the indicated cell lines at two different Effector:Target ratio.

FIG. 7. Polyfunctionality study of GPC3-2 and GPC3-1 CAR T constructs. scFvs are indicated on the left of each row, and co-stimulator domains are indicated at the top of each chart.

FIG. 8A and 8B. 8A. Effect of chimeric antigen receptor T cell (CAR T) transplant on body weight. Constructs used are shown in the legends at right. BW, body weight. ACT, adoptive T cell therapy. UT, untransduced T cells. PBS, phosphate-buffered saline. 7B. IHC depicting CAR-T accumulation in lung tissue in GPC3 CAR-T treated mice.

FIG. 9. Effect of administration of CAR T on tumor volume. Constructs used are shown in the legend at right. ACT, adoptive T cell therapy. UT, untransduced T cells. PBS, phosphate-buffered saline.

FIG. 10. Effect of administration of CAR T on survival. Constructs used are shown in the legend at right. ACT, adoptive T cell therapy. UT, untransduced T cells. PBS, phosphate-buffered saline.

FIGS. 11A-11D. Fluorescence-activated cell sorting (FACs) study of GPC3-1 CAR T cell differentiation and exhaustion using different co-stimulatory domains. Results for spleen (11A and 11B) and tumor cells (11C and 11D) are shown. Dot plots show the frequency of CD3+ T cells infiltrating each organ for each construct (11A and 11C). GPC3 CAR-T with 4-1BB/CD3 zeta (BZ) signaling domains are more central memory and less exhausted than CD28/CD3 zeta (28Z) in vivo. Co-stimulatory domains used are indicated at the top of each panel. Markers assayed are shown on the x and y axes. FSC, forward scatter. EM, effector memory. CM, central memory. TN, T naïve.

FIG. 12. GPC3-1 CAR T persistence in Hep3B and HepG2 tumors. Constructs used are shown in the legend at right. Percent CD3 is indicated. ACT, adoptive T cell therapy. UT, untransduced T cells.

FIG. 13. Effect of GPC3-1BZ treatment on body weight for non-tumor bearing and tumor bearing mice. Constructs used are shown in the legend at bottom. BW, body weight. ACT, adoptive T cell therapy. TZ is GPC3-1 TZ. UT, untransduced T cells. PBS, phosphate-buffered saline.

FIG. 14. Tumor volume and bleed timing for GPC3-1 BZ and GPC3-1 TZ cytokine analysis. Bleed points for the subsequent cytokine response study are indicated by arrows. Constructs used are shown in the legend at right. ACT, adoptive T cell therapy. UT, untransduced T cells. PBS, phosphate-buffered saline.

FIG. 15. Max systemic cytokine response (IFN-γ) GPC3-1 CAR T treatment. Data are shown for the bleed on day 8, on which the maximum cytokine response was observed. UT, untransduced T cells. PBS, phosphate-buffered saline.

FIG. 16. Histology of Hep3B tumor tissue in NOD scid gamma (NSG) immunodeficient mice. Top, untreated control. Bottom, animal treated with GPC3-1 BZ CAR T cells. Images are shown at 20×.

FIG. 17. Histology of intestinal nervous tissue in NOD scid gamma (NSG) immunodeficient mice. Left, untreated control. Right, animal treated with GPC3-1 BZ CAR T cells.

FIG. 18. Cell surface GPC3 quantification. From top to bottom, A375 cells (GPC3 negative), HepG2 cells (high GPC3), Hep3B cells (medium/low GPC3), and Huh7 cells (low GPC3). The areas under the peaks indicate the population of cells expressing the protein at the level indicated on the x axis. APC, allophycocyanin.

FIG. 19. Cytokine enzyme-linked immunosorbent assay (ELISA) results after 24-hour exposure to GPC3-1 BZ T cells. Cell lines are shown from left to right in the order listed from top to bottom in the legend.

FIG. 20. Immunohistochemistry against GPC3 for representative tumor xenografts from two HCC cell lines. Both xenografts scored as intensity =2. Data indicate tumors with at least 25% positive GPC3 expression at moderate intensity will respond to GPC3-1 BZ CAR-T.

FIG. 21. Determination of relative surface GPC3 expression. FSC, forward scatter. APC, allophycocyanin. MFI, mean fluorescence intensity. The frequency of GPC3 in each gate is shown in the dot plot on the left (12.7, 24, and 9.34%, respectively). The histogram on the right depicts the expression of GPC3 on the sorted populations, confirming the purity and homogeneity.

FIG. 22. Cytokine ELISA after 24-hour exposure to GPC3-1 BZ. Results are shown for T cells only, A375 cells (GPC3 negative), and low, medium (med), and high expressers of GPC3. Results are shown from left to right in each panel for each cell type in the order listed from top to bottom in the legend. UT, untransduced T cells. TZ and BZ, GPC3-1 TZ and GPC3-1 BZ.

FIG. 23. Interferon gamma (IFNγ) levels in different cell types after CAR T treatment. Constructs used are shown on the x axis. Results are shown from left to right for each construct for each cell type in the order listed from top to bottom in the legend. TZ, GPC3-1 TZ. UT, untransduced T cells. Medium, treatment with cell medium only.

FIG. 24. Cytokine levels in neuronal tissue cell types after treatment with GPC3-1 CAR T. Constructs used are shown on the x axis. Results are shown from left to right for each construct for each cell type in the order listed from top to bottom in the legend. UT, untransduced T cells. Medium, treatment with cell medium only.

FIG. 25. Tumor volume with treatment with CAR T and anti-CRS associated cytokine antibodies. CRS=cytokine release syndrome. Top, tumor volume upon treatment with different schemes of CARs and antibodies. Bottom, studies of individual subjects upon treatment with GPC3-1 BZ+PBS, GPC3-1 BZ+anti-IL-6, and GPC3-1 BZ+anti-TNF-α. MEDI7028 is GPC3-1 BZ. ACT, adoptive T cell therapy. UT, untransduced T cells. PBS, phosphate-buffered saline.

FIGS. 26A-26C. Study of higher CAR T doses and anti-TNFα treatment in a resistant HCC model (Huh7). Anti-TNFα can be used to attenuate toxicity and promote anti-tumor activity at higher CAR-T doses. Constructs used are shown in the legend at right. 26A. Study schema. BW, body weight. 26B. Tumor growth. i.v., intravenous. 26C. Change in body weight.

### DETAILED DESCRIPTION

### 1. Definitions

Unless defined otherwise, all technical and scientific terms used herein have the meaning commonly understood by a person skilled in the art to which this invention belongs. The following references provide one of skill with a general definition of many of the terms used in this invention: Singleton, et al., Dictionary of Microbiology and Molecular Biology (2nd ed. 1994); The Cambridge Dictionary of Science and Technology (Walker ed., 1988); The Glossary of Genetics, 5th Ed., R. Rieger, et al. (eds.), Springer Verlag (1991); and Hale & Marham, The Harper Collins Dictionary of Biology (1991). As used herein, the following terms have the meanings ascribed to them below, unless specified otherwise.

As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indictates otherwise.

Percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. The term “about” also includes the exact amount. For example, “about 5%” means “about 5%” and also “5%.” The term “about” can also refer to ±10% of a given value or range of values. Therefore, about 5% also means 4.5%-5.5%, for example. Unless otherwise clear from context, all numerical values provided herein are modified by the term “about.”

As used herein, the terms “or” and “and/or” can describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “polypeptide” refers to a molecule composed of monomers (amino acids) linearly linked by amide bonds (also known as peptide bonds). The term “polypeptide” refers to any chain or chains of two or more amino acids. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

An “isolated” substance, e.g., isolated nucleic acid, is a substance that is not in its natural milieu, though it is not necessarily purified. For example, an isolated nucleic acid is a nucleic acid that is not produced or situated in its native or natural environment, such as a cell. An isolated substance can have been separated, fractionated, or at least partially purified by any suitable technique.

As used herein, the terms “antibody” and “antigen-binding fragment thereof” refer to at least the minimal portion of an antibody which is capable of binding to a specified antigen which the antibody targets, e.g., at least some of the complementarity determining regions (CDRs) of the variable domain of a heavy chain (VH) and the variable domain of a light chain (VL) in the context of a typical antibody produced by a B cell. Antibodies or antigen-binding fragments thereof can be or be derived from polyclonal, monoclonal, human, humanized, or chimeric antibodies, single chain antibodies, epitope-binding fragments, e.g., Fab, Fab′ and F(ab′)2, Fd, Fvs, single-chain Fvs (scFvs), single-chain antibodies, disulfide-linked Fvs (sdFvs), fragments comprising either a VL or VH domain alone or in conjunction with a portion of the opposite domain (e.g., a whole VL domain and a partial VH domain with one, two, or three CDRs), and fragments produced by a Fab expression library. ScFv molecules are known in the art and are described, e.g., in U.S. Pat. No. 5,892,019. Antibody molecules encompassed by this disclosure can be of or be derived from any type (e.g., IgG, IgE, IgM, IgD, IgA, and IgY), class (e.g., IgG1, IgG2, IgG3, IgG4, IgA1 and IgA2), or subclass of immunoglobulin molecule.

As used herein, the term “polynucleotide” includes a singular nucleic acid as well as multiple nucleic acids, and refers to an isolated nucleic acid molecule or construct, e.g., messenger RNA (mRNA) or plasmid DNA (pDNA). The term “nucleic acid” includes any nucleic acid type, such as DNA or RNA.

As used herein, the term “vector” can refer to a nucleic acid molecule as introduced into a host cell, thereby producing a transformed host cell. A vector can include nucleic acid sequences that permits it to replicate in a host cell, such as an origin of replication. A vector can also include one or more selectable marker gene and other genetic elements known in the art. Specific types of vector envisioned here can be associated with or incorporated into viruses to facilitate cell transformation.

A “transformed” cell, or a “host” cell, is a cell into which a nucleic acid molecule has been introduced by molecular biology techniques. All techniques by which a nucleic acid molecule can be introduced into such a cell, including transfection with viral vectors, transformation with plasmid vectors, and introduction of naked DNA by electroporation, lipofection, and particle gun acceleration are contemplated herein.

As used herein, the term “affinity” refers to a measure of the strength of the binding of a antigen or target (such as an eptitope) to its cognate binding domain (such as a paratope). As used herein, the term “avidity” refers to the overall stability of the complex between a population of epitopes and paratopes (i.e., antigens and antigen binding domains).

As used herein, the terms “treat,” “treatment,” or “treatment of” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on.

As used herein, the term an “effective amount” or a “therapeutically effective amount” of an administered therapeutic substance, such as a CAR T cell, is an amount sufficient to carry out a specifically stated or intended purpose, such as treating or treatment of cancer. An “effective amount” can be determined empirically in a routine manner in relation to the stated purpose.

### 2. Overview

The present disclosure is directed to compositions and methods for treating cancer using chimeric antigen receptor (CAR) cell therapy. More particularly, the present disclosure concerns CAR cell therapies in which the transformed cells, such as T cells, express CARs that target Glypican-3 (GPC3). Still further, the CAR constructs, transformed cells expressing the constructs, and the therapies utilizing the transformed cells disclosed herein can provide robust cancer treatments with minimized risk of cytokine release syndrome (CRS) or indiscriminate cytokine release in non-GPC3 expressing cells.

Without wishing to be bound by theory, GPC3 is believed to be a viable cancer target across multiple modalities, including bispecific T cell engagers, CAR cells, as well as monoclonal antibodies and antibody-drug conjugates (ADCs). GPC3, an onco-fetal antigen, is a GPI-linked heparin sulfate proteoglycan. GPC3 stabilizes the Wnt-Fzd interaction, stimulating Wnt signaling. GPC3 competes with Patched for Hh binding, relieving Smoothened inhibition, and inducing GPC3 degradation. Both pathways have been shown to stimulate hepatocellular carcinoma (HCC) growth. And, GPC3 expression levels have been shown to correlate with stage and grade of HCC.

Further, it is believed that GPC3 is a promising target for CAR cell therapy. Therefore, antibodies and CAR constructs derivized from these antibodies have been developed as described herein.

### 3. CAR Construct Design

CAR constructs of the present disclosure can have several components, many of which can be selected based upon a desired or refined function of the resultant CAR construct. In addition to an antigen binding domain, CAR constructs can have a spacer domain, a hinge domain, a signal peptide domain, a transmembrane domain, and one or more costimulatory domains. Selection of one component over another (i.e., selection of a specific co-stimulatory domain from one receptor versus a co-stimulatory domain from a different receptor) can influence clinical efficacy and safety profiles.

### 4. Antigen Binding Domain

Antigen binding domains contemplated herein can include antibodies or one or more antigen-binding fragments thereof. One contemplated CAR construct targeting GPC3 comprises a single chain variable fragment (scFv) containing light and heavy chain variable regions from one or more antibodies specific for GPC3 that are either directly linked together or linked together via a flexible linker (e.g., a repeat of GGGGS having 1, 2, 3 or more repeats).

The antigen binding domain of a CAR targeting GPC3 as disclosed herein can vary in its binding affinity for the GPC3 protein. The relationship between binding affinity and efficacy can be more nuanced in the context of CARs as compared with antibodies, for which higher affinity is typically desirable. For example, preclinical studies on a receptor tyrosine kinase-like orphan receptor 1 (ROR1)-CAR derived from a high-affinity scFv (with a dissociation constant of 0.56 nM) resulted in an increased therapeutic index when compared with a lower-affinity variant. Converserly, other examples have been reported that engineering the scFv for lower affinity improves the discrimination among cells with varying antigen density. This could be useful for improving the therapeutic specificity for antigens differentially expressed on tumor versus normal tissues.

A variety of methods can be used to ascertain the binding affinity of the antigen binding domain. In some embodiments, methodologies that exclude avidity effects can be used. Avidity effects involve multiple antigen-binding sites simultaneously interacting with multiple target epitopes, often in multimerized structures. Thus, avidity functionally represents the accumulated strength of multiple interactions. An example of a methodology that excludes avidity effects is any approach in which one or both of the interacting proteins is monomeric/monovalent since multiple simultaneous interactions are not possible if one or both partners contain only a single interaction site.

### 5. Spacer Domain

A CAR construct of the present disclosure can have a spacer domain to provide conformational freedom to facilitate binding to the target antigen on the target cell. The optimal length of a spacer domain may depend on the proximity of the binding epitope to the target cell surface. For example, proximal epitopes can require longer spacers and distal epitopes can require shorter ones. Besides promoting binding of the CAR to the target antigen, achieving an optimal distance between a CAR cell and a cancer cell may also help to sterically occlude large inhibitory molecules from the immunological synapse formed between the CAR cell and the target cancer cell. A CAR targeting GPC3 can have a long spacer, an intermediate spacer, or a shorter spacer. Long spacers can include a CH2CH3 domain (˜220 amino acids) of immunoglobulin G1 (IgG1) or IgG4 (either native or with modifications common in therapeutic antibodies, such as a S228P mutation), whereas the CH3 region can be used on its own to construct an intermediate spacer (˜120 amino acids). Shorter spacers can be derived from segments (<60 amino acids) of CD28, CD8a, CD3 or CD4. Short spacers can also be derived from the hinge regions of IgG molecules. These hinge regions may be derived from any IgG isotype and may or may not contain mutations common in therapeutic antibodies such as the S228P mutation mentioned above.

### 6. Hinge Domain

A CAR targeting GPC3 can also have a hinge domain. The flexible hinge domain is a short peptide fragment that provides conformational freedom to facilitate binding to the target antigen on the tumor cell. It may be used alone or in conjunction with a spacer sequence. The terms “hinge” and “spacer” are often used interchangably—for example, IgG4 sequences can be considered both “hinge” and “spacer” sequences (i.e., hinge/spacer sequences).

A CAR targeting GPC3 can further include a sequence comprising a signal peptide. Signal peptides function to prompt a cell to translocate the CAR to the cellular membrane. Examples include an IgG1 heavy chain signal polypeptide, Ig kappa or lambda light chain signal peptides, granulocyte-macrophage colony stimulating factor receptor 2 (GM-CSFR2 or CSFR2) signal peptide, a CD8a signal polypeptide, or a CD33 signal peptide.

### 7. Transmembrane Domain

A CAR targeting GPC3 can further include a sequence comprising a transmembrane domain. The transmembrane domain can include a hydrophobic a helix that spans the cell membrane. The properties of the transmembrane domain have not been as meticulously studied as other aspects of CAR constructs, but they can potentially affect CAR expression and association with endogenous membrane proteins. Transmembrane domains can be derived, for example, from CD4, CD8α, or CD28.

### 8. Costimulatory Domain

A CAR targeting GPC3 can further include one or more sequences that form a co-stimulatory domain. A co-stimulatory domain is a domain capable of potentiating or modulating the response of immune effector cells. Co-stimulatory domains can include sequences, for example, from one or more of CD3zeta (or CD3z), CD28, 4-1BB, OX-40, ICOS, CD27, GITR, CD2, IL-2Rβ and MyD88/CD40. The choice of co-stimulatory domain influences the phenotype and metabolic signature of CAR cells. For example, CD28 co-stimulation yields a potent, yet short-lived, effector-like phenotype, with high levels of cytolytic capacity, interleukin-2 (IL-2) secretion, and glycolysis. By contrast, T cells modified with CARs bearing 4-1BB costimulatory domains tend to expand and persist longer in vivo, have increased oxidative metabolism, are less prone to exhaustion, and have an increased capacity to generate central memory T cells.

### 9. Cells

CAR-based cell therapies can be used with a variety of cell types, such as lymphocytes. Particular types of cells that can be used include T cells, Natural Killer (NK) cells, Natural Killer T (NKT) cells, Invariant Natural Killer T (iNKT) cells, alpha beta T cells, gamma delta T cells, viral-specific T (VST) cells, cytotoxic T lymphocytes (CTLs), and regulatory T cells (Tregs). In one embodiment, CAR cells for treating a subject are autologous. In other embodiments, the CAR cells may be from a genetically similar, but non-identical donor (allogeneic).

### 10. CAR Cell Production

CAR constructs of the present disclosure can include some combination of the modular components described herein. For example, in some embodiments of the present disclosure, a CAR construct comprises a GPC3-1 scFv antigen binding domain. In some embodiments, a CAR comprises a GPC3-2 scFv antigen binding domain. In some embodiments of the present disclosure, a CAR construct comprises a CSFR2 signal peptide. In some embodiments, a CAR construct comprises an IgG4P hinge/spacer domain carrying an S228P mutation. In some embodiments, a CAR construct comprises a CD28 transmembrane.

Different co-stimulatory domains can be utilized is the CAR constructs of the present disclosure. In some embodiments, a CAR construct comprises a co-stimulatory domain from the intracellular domain of CD3z. In some embodiments, a CAR construct comprises a CD28 co-stimulatory domain. In some embodiments, a CAR construct comprises a 4-1BB co-stimulatory domain. In some embodiments, a CAR construct comprises co-stimulatory domains from CD3z and CD28. In some embodiments, a CAR construct comprises co-stimulatory domains from CD3z and 4-1BB. In some embodiments, a CAR construct comprises co-stimulatory domains from all of CD3z, CD28, and 4-1BB. In some embodiments, a CAR construct comprises co-stimulatory domains from ICOS, OX-40, and/or GITR.

### 11. CAR Construct Assessment

Constructs of the present disclosure were compared and assessed based on safety as well as persistence and establishment of central memory. The lower affinity (high off-rate) scFv, GPC3-1, was assessed favorably on account of its improved safety. The 4-1BB and CD3z co-stimulatory domains (both in the same construct) were assessed favorably based on their contribution to improved persistence and favorable in vivo phenotype (more central memory). The GPC3-1 and GPC3-2 CARs of the present disclosure compared favorably to constructs based on published GPC3-targeting CARs. Details of the assessment can be found in the Examples.

### 12. Embodiments

In some embodiments, the present disclosure provides an isolated nucleic acid sequence encoding a chimeric antigen receptor (CAR). The CAR comprises an antigen binding domain specific for glypican 3 (GPC3). The antigen binding domain has an equilibrium dissociation constant (K_{D}) of about 100 nanomolar (nM) or less, and the CAR construct does not induce cytokine production in GPC3-cells. In some embodiments, the antigen binding domain includes an antibody or antigen-binding fragment thereof. The antigen binding domain can be a Fab or a single chain variable fragment (scFv). In some embodiments, the antigen binding domain is an scFv comprising the nucleic acid sequence of SEQ ID NO: 33 or SEQ ID NO: 34.

In some embodiments, the CAR further includes a transmembrane domain, a costimulatory domain, and a signal domain. The transmembrane domain can be a CD28 transmembrane domain. The costimulatory domain can be one or more of CD28, 4-1BB, CD3zeta, OX-40, ICOS, CD27, GITR, and MyD88/CD40 costimulatory domains. In one specific embodiment. the costimulatory domain is one or more of CD28, 4-1BB, and CD3zeta costimulatory domains. The signal domain can be a sequence encoding a CSFR2 signal peptide.

In some embodiments, the isolated nucleic acid sequence can include a hinge/spacer domain. The hinge/spacer domain can be an IgG4P hinge/spacer.

In some specific embodiments, an isolated nucleic acid sequence encoding a chimeric antigen receptor (CAR) can have the sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 26.

In other embodiments, the present disclosure provides an anti-GPC3 chimeric antigen receptor (CAR) including an antigen binding domain. The antigen binding domain can be an antibody, Fab, or an scFv comprising a heavy chain variable region (VH) and a light chain variable region (VL). In some embodiments, the VH can have a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39. In some embodiments, the VL can have a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

In some embodiments, the VH can be the amino acid sequence of SEQ ID NO: 27 or SEQ ID NO: 29, and the VL can be the amino acid sequence of SEQ ID NO: 28 or SEQ ID NO: 30. In some embodiments, the CAR further can have a transmembrane domain, a costimulatory domain, and a signal domain.

In some specific embodiments, the anti-GPC3 CAR can have the amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25.

In other embodiments, the present disclosure provides a vector comprising a nucleic acid sequence encoding a chimeric antigen receptor (CAR). The nucleic acid sequence can be SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

In other embodiments, the present disclosure provides a cell comprising a vector having a nucleic acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

In other embodiments, the present disclosure provides a cell having a nucleic acid sequence encoding a chimeric antigen receptor (CAR), wherein the CAR comprises an antigen binding domain specific for glypican 3 (GPC3), wherein the antigen binding domain has an equilibrium dissociation constant (K_{D}) of about 100 nanomolar (nM) or less, and wherein the CAR construct does not induce cytokine production in GPC3-cells. For example, the nucleic acid sequence can be SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

In other embodiments, the present disclosure provides a cell expressing an anti-GPC3 chimeric antigen receptor (CAR) on a extracellular surface thereof. The CAR can have an antigen binding domain that can be an antibody, a Fab, or an scFv each having a heavy chain variable region (VH) and a light chain variable region (VL). The VH can include a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39. The VL can include a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

In some embodiments, the VH can have the amino acid sequence of SEQ ID NO: 27 or SEQ ID NO: 29. In some embodiments, the VL can have the amino acid sequence of SEQ ID NO: 28 or SEQ ID NO: 30. The CAR can further include a transmembrane domain, a costimulatory domain, and a signal domain. The cell express a CAR having an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25.

In some embodiments, the present disclosure provides a T cell, a Natural Killer (NK) cell, a cytotoxic T lymphocyte (CTL), and/or a regulatory T cell that express a CAR on an extracellular surface thereof, and the CAR can have an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25. Such cells can exhibit an anti-tumor immunity upon contacting a tumor cell expressing GPC3.

### 13. Treatment of Cancers With CARs

In some embodiments, the present disclosure provides CAR cells for treatment of cancer. The compositions (e.g., antibodies, CAR constructs, and CAR cells) and methods of their use described herein are especially useful for inhibiting neoplastic cell growth or spread; particularly neoplastic cell growth in which GPC3 plays a role.

Neoplasms treatable by the compositions of the disclosure include solid tumors, for example, those of the liver, lung, or ovary. However, the cancers listed herein are not intended to be limiting. For example, types of cancer that are contemplated for treatment herein include, for example, NSCLC, advanced solid malignancies, biliary tract neoplasms, bladder cancer, colorectal cancer, diffuse large b-cell lymphoma, esophageal neoplasms, esophageal squamous cell carcinoma, extensive stage small cell lung cancer, gastric adenocarcinoma, gastric cancer, gastroesophageal junction cancer, head and neck cancer, head and neck squamous cell carcinoma, hepatocellular carcinoma, Hodgkin lymphoma, lung cancer, melanoma, mesothelioma, metastatic clear cell renal carcinoma, metastatic melanoma, metastatic non-cutaneous melanoma, multiple myeloma, nasopharyngeal neoplasms, non-Hodgkin lymphoma, ovarian cancer, fallopian tube cancer, peritoneal neoplasms, pleural mesothelioma, prostatic neoplasms, recurrent or metastatic PD-L1 positive or negative SCCHN, recurrent squamous cell lung cancer, renal cell cancer, renal cell carcinoma, SCCHN, hypo pharyngeal squamous cell carcinoma, laryngeal squamous cell carcinoma, small cell lung cancer, squamous cell carcinoma of the head and neck, squamous cell lung carcinoma, TNBC, transitional cell carcinoma, unresectable or metastatic melanoma, urothelial cancer, and urothelial carcinoma.

In one embodiment, cancers contemplated for treatment here include any that express GPC3 on the cell surfaces of the cancer cells. In one specific example, cancers contemplated for treatment herein include hepatocellular carcinoma, non-small cell lung cancer, ovarian cancer, and squamous cell lung carcinoma.

### 14. Methods of Treatment

CAR-modified cells of the present invention, such as CAR T cells, may be administered alone or as a pharmaceutical composition with a diluent and/or other components associated with cytokines or cell populations. Briefly, pharmaceutical compositions of the invention can include, for example CAR T cells as described herein, with one or more pharmaceutically or physiologically acceptable carrier, diluent, or excipient. Such compositions can comprise buffers such as neutral buffered saline, buffered saline, and the like; sulfates; carbohydrates such as glucose, mannose, sucrose, or dextrans, mannitol; proteins, polypeptides, or amino acids such as glycine; antioxidants; chelating agents such as EDTA or glutathione; adjuvants (e.g., aluminum hydroxide); and preservatives. The pharmaceutical compositions of the invention may be adapted to the treatment (or prophylaxis).

The CAR-modified cells can also be administered in conjunction with one or more additional therapies. In one embodiment, the additional therapies can include anti-cytokine antibodies. For example, one or more anti-TNFα antibodies can be used to attenuate toxicity and promote anti-tumor activity at higher CAR T doses, which can be associated with CRS-like symptoms and weight loss.

In a particular embodiment, a treatment regimen contemplated can include one or more biological components, such as a CAR T cell and an anticancer antibody and/or a chemotherapeutic component. For example, it is contemplated that a treatment regimen can additionally include an immune checkpoint inhibitor (ICI), such as those that target the PD-1/PD-L1 axis (PDX) and other immune-oncology (IO) treatments, such as immune system agonists.

Contemplated antibodies include an anti-PD-L1 antibody such as durvalumab (MEDI4736), avelumab, atezolizumab, KNO35, an anti-PD-1 antibody such as nivolumab, pembrolizumab, REGN2810, SHR1210, IBI308, PDR001, Anti-PD-1, BGB-A317, BCD-100, and JS001, and an anti-CTLA4 antibody, such as tremelimumab or ipilimumab. Additional antibodies are also contemplated herein. Any therapeutically effective antibody subparts are also contemplated herein.

Information regarding durvalumab (or fragments thereof) for use in the methods provided herein can be found in U.S. Pat. Nos. 8,779,108; 9,493,565; and 10,400,039 the disclosures of which are incorporated herein by reference in its entirety. In a specific aspect, durvalumab or an antigen-binding fragment thereof for use in the methods provided herein comprises the variable heavy chain and variable light chain CDR sequences of the 2.14H9OPT antibody as disclosed in the aforementioned U.S. patents.

Information regarding tremelimumab (or antigen-binding fragments thereof) for use in the methods provided herein can be found in U.S. Pat. No. 6,682,736 (in which tremelimumab is referred to as 11.2.1), the disclosure of which is incorporated herein by reference in its entirety.

Additional therapeutics (chemotherapies or biologics) contemplated herein include without limitation cisplatin/gemcitabine or methotrexate, vinblastine, ADRIAMYCIN™ (doxorubicin), cisplatin (MVAC), carboplatin-based regimen, or single-agent taxane or gemcitabine, temozolomide, or dacarbazine, vinflunine, docetaxel, paclitaxel, nab-paclitaxel, Vemurafenib, Erlotinib, Afatinib, Cetuximab, Bevacizumab, Erlotinib, Gefitinib, and/or Pemetrexed. Further examples include drugs targeting DNA damage repair systems, such as poly (ADP-ribose) polymerase 1 (PARP1) inhibitors and therapeutics inhibiting WEE1 protein kinase activity, ATR protein kinase activity, ATM protein kinase activity, Aurora B protein kinase activity, and DNA-PK activity.

Any therapeutic compositions or methods contemplated herein can be combined with one or more of any of the other therapeutic compositions and methods provided herein.

In some embodiments, the present disclosure provides a method of treating cancer including administering to a subject in need thereof an effective amount of a cell comprising an anti-GPC3 chimeric antigen receptor (CAR) comprising an antigen binding domain. The antigen binding domain can be an antibody, Fab, or an scFv comprising a heavy chain variable region (VH) and a light chain variable region (VL). The VH can include a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39. The VL can include a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45. In some embodiments, the method further inhibits tumor growth, induces tumor regression, and/or prolongs survival of the subject.

In some embodiments, the cell is an autologous cell. For example, the autologous cell can be selected from the group consisting of a T cell, a Natural Killer (NK) cell, a cytotoxic T lymphocyte (CTL), and a regulatory T cell.

In some embodiments, the cancer treated by the method is a solid tumor. For example, the cancer can be hepatocellular carcinoma, non-small cell lung cancer, ovarian cancer, and/or squamous cell lung carcinoma. In a specific embodiment, the cancer is hepatocellular carcinoma.

the present disclosure provides a method of treating cancer including administering to a subject in need thereof an effective amount of a cell comprising an anti-GPC3 chimeric antigen receptor (CAR) and an effective amount of an anti-TNFα antibody.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

### EXAMPLES

The Examples that follow are illustrative of specific embodiments of the disclosure, and various uses thereof. They are set forth for explanatory purposes only and should not be construed as limiting the scope of the disclosure in any way. A description of terms is provided in Table 1.

TABLE 1
Description of terms

| Term | Description |
|---|---|
| GPC3-1 | Anti-GPC3 scFv (lower affinity) |
| GPC3-2 | Anti-GPC3 scFv (higher affinity) |
| GPC3-4 | Previously disclosed anti-GPC3 scFv (UPenn) |
| GPC3-3 | Previously disclosed anti-GPC3 scFv (Baylor) |
| BZ | Intracellular domain of CAR with two co-stimulatory |
| | domains of 4-1BB and CD3zeta |
| TZ | Intracellular domain of CAR with a truncated CD3zeta |
| | signaling domain (acting as a signaling incompetent |
| | control) |
| 28Z | Intracellular domain of CAR with two co-stimulatory |
| | domains of CD28 and CD3zeta |
| 28BZ | Intracellular domain of CAR with three co-stimulatory |
| | domains of CD28, 4-1BB, and CD3zeta |
| GPC3-1 BZ | CAR with GPC3-1 scFv, CSFR2 signal peptide, IgG4P |
| (MEDI7028) | hinge region, and both 4-1BB and CD3z co-stimulatory |
| | domains |
| GPC3-2 BZ | CAR with GPC3-2 scFv, CSFR2 signal peptide, IgG4P |
| | hinge region, and both 4-1BB and CD3z co-stimulatory |
| | domains |
| CSFR2 | Signal peptide used in all CAR constructs |
| IgG4P | Hinge sequence used in all CAR constructs |
| Hep3B | Hepatocellular carcinoma model |
| Huh7 | Resistant hepatocellular carcinoma (HCC) model |

### Example 1: Expression of GPC3

### Methods

GPC3 IHC utilized the mouse monoclonal anti-human GPC3 antibody GC33 (Ventana). Secondary staining was performed using anti-mouse HRP. Human tissue micro-arrays (TMAs, US Biomax), representing hepatocellular carcinoma (HCC), non-small cell lung cancer (NSCLC), and ovarian cancer, or human colonic ganglion tissue, were stained for GPC3 expression and staining intensity and pattern were determined by microscopy.

### Results

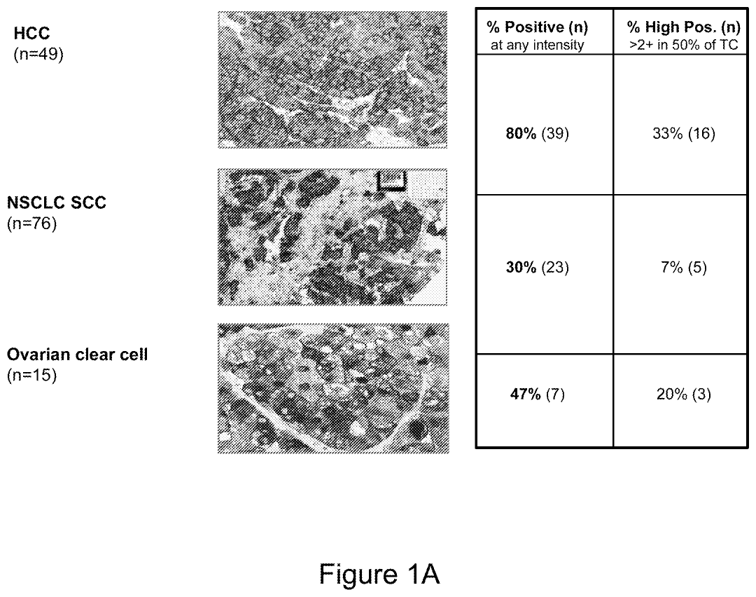

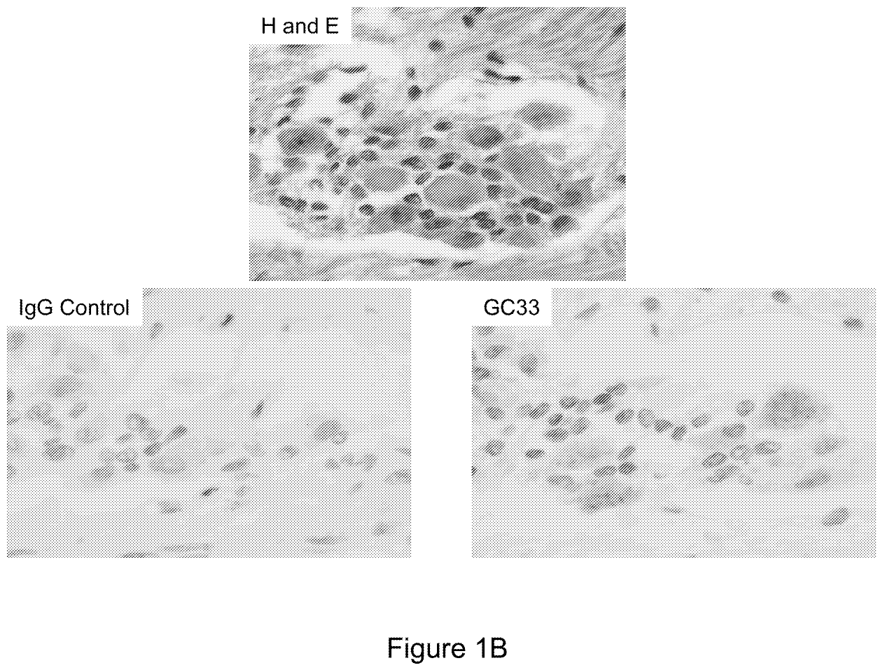

GPC3 is overexpressed in 80% of HCC, 30% of squamous lung carcinoma, and 47% of ovarian clear cell carcinoma. However, GPC3 is not detectable by immunohistochemistry in normal liver tissue, including cirrhotic and hyperplastic samples, and has low expression in normal tissues (e.g., lung). See FIGS. 1A and 1B.

### Example 2: Development and Affinity Studies of scFvs

### Summary

In the present example, anti-GPC3 scFvs were developed and their relative affinities for GPC3 were determined.

### Methods

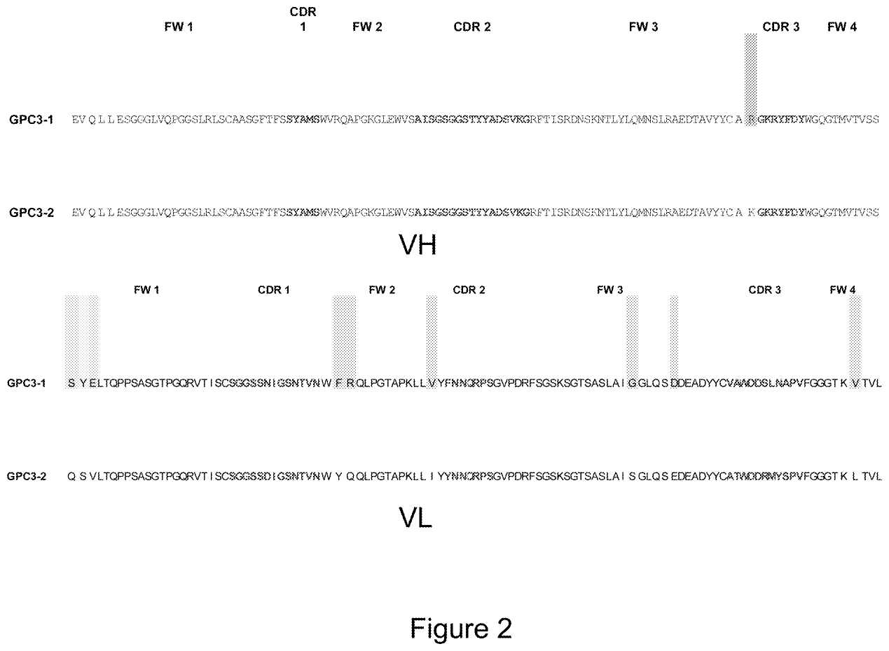

GPC3-1 (SEQ ID NO: 1) and GPC3-2 (SEQ ID NO: 2) have a nearly identical V_{H} domain (SEQ ID NOS: 27 and 29), but different V_{L} domain (SEQ ID NOS: 28 and 30; see FIG. 2). GPC3-2 has a fully germline framework, whereas GPC3-1 does not.

Apparent binding affinity was determined by cell surface binding of soluble recombinant GPC3 protein to the GPC3-1 and GPC3-2 CARs expressed on the surface of Jurkat cells. CAR constructs were expressed on the surface of Jurkat cells using lentiviral vectors. Cells were stained with varying concentrations of recombinant His-tagged GPC3 protein (R&D systems). Bound GPC3 was visualized by staining with a fluorescently conjugated anti-His-tag secondary antibody, and cells were analyzed by flow cytometry. Binding curves were fit to a simple one site binding model to determine the apparent K_{D}.

An alternative measure of binding affinity was determined using a BIAcore surface plasmon resonance system and GPC3-1 and GPC3-2 scFv-Fc fusion proteins. Purified scFv-Fc fusion molecules of GPC3-1 and GPC3-2 were covalently coupled to an amine reactive SPR sensor chip (CMS, GE Healthcare). For GPC-1, soluble GPC3 protein (R&D Systems) was flowed over the chip surface at concentrations of 14, 28, 57, 114, and 228 nM at a rate of 30 μL/min, and the interaction was monitored. For GPC3-2, concentrations of 4, 7, 14, 28, and 57 nM were flowed at the same flow rate. Data were fit using BIAevaluation software (GE Healthcare), and a simple 1:1 Langmuir binding model, with Rₘₐₓ fit globally and kₐ, k_{d}, and K_{D} fit locally.

### Results

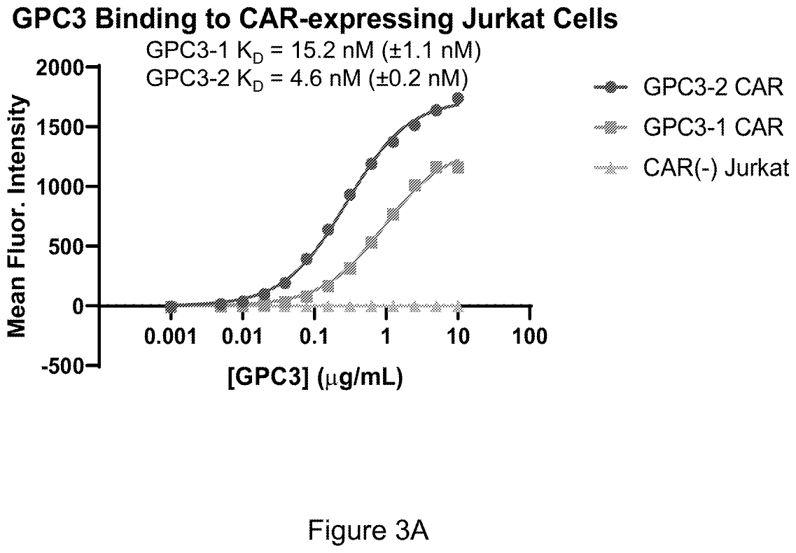

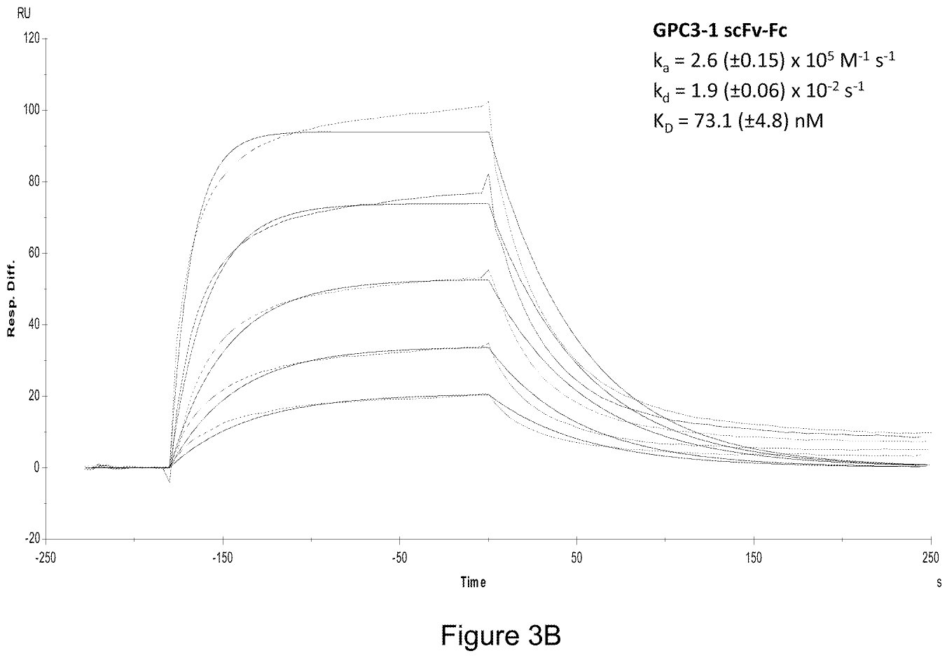

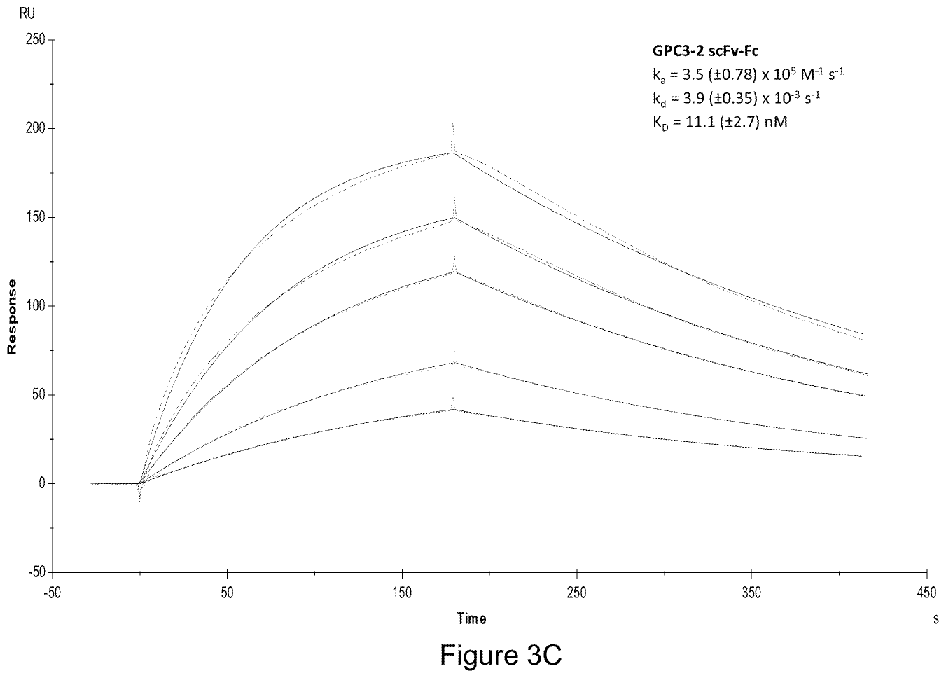

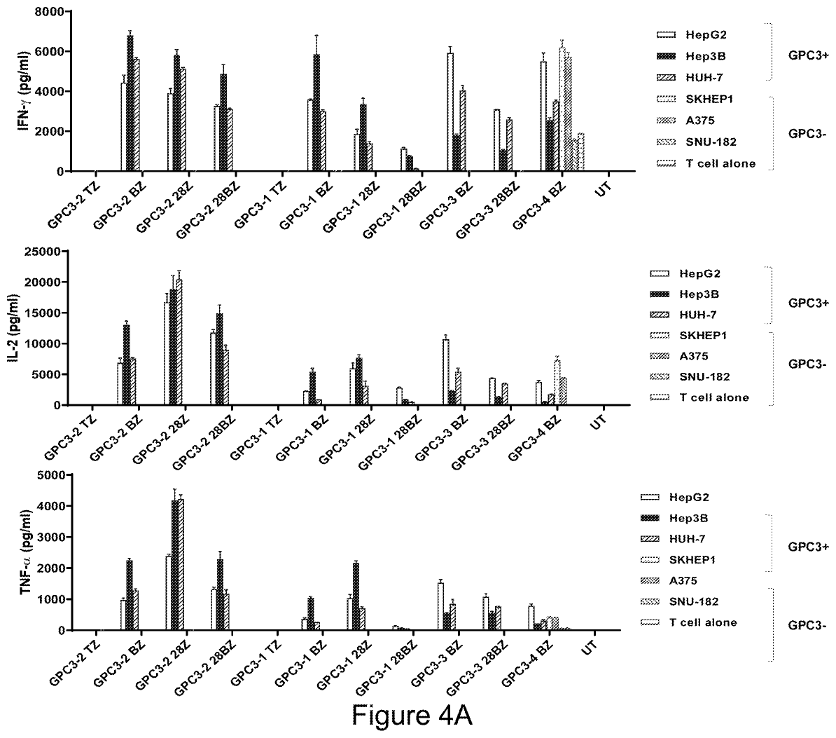

In experiments assessing soluble GPC3 binding to GPC3-1 and GPC3-2 CARs expressed on the surface of Jurkat cells, K_{d} values were approximately 15 nM for GPC3-1 and 5 nM for GPC3-2 (see FIG. 3A). In surface plasmon resonance experiments using the same GPC3 protein as used for cell-based binding and purified GPC3-1/GPC3-2 scFv-Fc fusion proteins, K_{D} values for GPC3-1 and GPC3-2 were approximately 73 nM and 11 nM, respectively. See Table 1 and FIG. 3B and 3C.

TABLE 1
Monovalent binding values.

| | Monovalent Binding (ProteOn) Soluble HuGPC3 | | |
|---|---|---|---|
| Clone Name | kon (M⁻¹S¹) | koff (S¹) | K_{D} (nM) |
| GPC3-1 | 2.6 × 10⁵ | 1.9 × 10⁻² | 73 |
| GPC3-2 | 3.5 × 10⁵ | 3.9 × 10⁻³ | 11 |

Reported Kd values for four scFvs are shown in Table 2.

TABLE 2
Dissociation constants (K_{d}).

| scFv | K_{d} (nM) |
|---|---|
| GPC3-1 | 73 |
| GPC3-2 | 11 |
| GPC3-3 | 0.5 |
| GPC3-4 | 12 |

### Example 3. Development and in Vitro Study of CAR Constructs

### Summary

In the present example, anti-GPC3 CAR constructs were developed and tested for resultant cytokine activity and polyfunctionality.

### Methods

Structure of CARs. For all CAR constructs, a CSFR2 signal peptide (used in a number of clinical stage CAR T constructs) was used. An IgG4P (S228P mutation) hinge domain was used as the “spacer” between the scFv and the membrane, and a CD28 transmembrane domain was utilized. On the intracellular side, different co-stimulatory domains were tested, including varied combinations of CD28, 4-1BB, and CD3zeta co-stimulatory domains. Constructs using co-stimulatory domains from inducible T cell co-stimulator (ICOS), OX40, and glucocorticoid-induced TNFR family related gene (GITR) were also attempted. Sequences for the GPC3-1 and GPC3-2 CAR constructs are shown in SEQ ID NOS: 3-10, and the corresponding nucleic acid sequences are shown in SEQ ID NOS: 11-18.

Other known CARs against GPC3 (based on GPC3-3 and GPC3-4 scFvs) were constructed for comparison against the GPC3-1 and GPC3-2 CAR constructs. The GPC3-3 CAR contained a short IgG1 hinge, a CD28 transmembrane domain, a 4-1BB co-stimulatory domain, and a CD3zeta intracellular domain. Another GPC3-3 CAR construct that has both CD28 and 4-1BB co-stimulatory domains was also developed. The GPC3-4 CAR construct comprised an IgG4P hinge, a CD28 transmembrane domain, and a 4-1BB co-stimulatory domain. Sequences for the GPC3-3 CAR and the GPC3-4 CAR are shown in SEQ ID NOS: 19-21, and the corresponding nucleic acid sequences are shown in SEQ ID NOS: 22-24.

CAR T cell production. Purified human T cells were seeded in AIM-V media containing 5% human serum and 1% penicillin-streptomycin, at a concentration of 0.2×10⁶ cells/mL+IL-2 (300 IU/mL). T cells were activated with anti-CD3/anti-CD28 Dynabeads (Invitrogen), and 24 hours later, transduction was performed by spinoculation. Lentivirus was added to the wells (M.O.I. 100) and plates were centrifuged at 2000 rpm, at 37° C. for 2 hours and placed in a 37° C., 5% CO₂ incubator. Cells were split as necessary to maintain cell density at approximately 0.5-1×10⁶ cells/mL. CAR-T cells were immunophenotyped not before 7 days post-transduction and assessed in in vitro and in vivo functional assays at around 11 days post-transduction.

Cell line testing. A variety of cell types were treated with CAR T cells having different CAR constructs. For all cytokine studies, 5×10⁴ CAR-T cells were co-cultured with target cells at a 1:1 ratio in RPMI 10% FCS. 24 hours later, supernatants were collected. Cytokines were analyzed by Meso Scale Discovery 4-plex Kit to detect IFN-γ, IL-2, TNF-α, and IL-10. Concentrations of cytokines in picograms per milliliter were determined.

Cytotoxity studies were performed using cellular impedance monitoring technology (xCELLigence). 3×10⁴ target cells were plated, and CAR-T cells were added 24 hours later at an effector to target (E:T) ratio of 3, 1, or 0.3. A normalized cell index was determined for Hep3B, Huh7, and SNU-182 cells after treatment with various CAR constructs. Hep3B expresses high GPC3 (14 k/cell), Huh7 expresses medium/low GPC3 (7 k/cell), and SNU-182 is negative for GPC3 (0/cell).

A polyfunctionality study was also performed on the CAR constructs. Here, GPC3-1 BZ or the indicated CAR-T cells were co-cultured for 6 hours with Hep3B or A375 in presence of Golgi Stop and a fluorophore labeled antibody against the degranulation marker CD107a. Target engagement induced CAR-T degranulation, and consequent binding of the fluorescently labeled anti-CD107 present in the culture medium. CD107 accumulation detected by flow cytometry is directly proportional to the extent of the degranulation and indicates lysis of the target cell. Because cells were incubated in the presence of Golgi Stop, the production of effector cytokines (IFN-γ, IL-2, TNF-α) could also be evaluated by intracellular staining. Boolean gates combining each function (CD107α, IFN-γ, IL-2, and TNF-a) were generated with Flowtop and pie charts of results were generated with Spice analysis software.

### Results

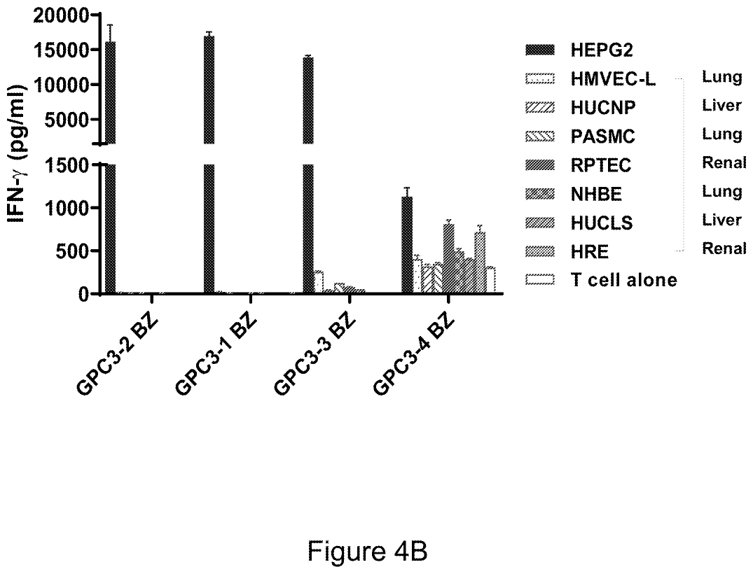

An overall higher degree of TNFα and IL-2 output for GPC3-2 and GPC3-3 constructs versus GPC3-1 was observed. Treatment with CAR T cells with GPC3-1 and GPC3-2 CAR constructs yielded antigen-specific cytokine production. On the other hand, the GPC3-4 BZ construct induced cytokines even in cell types that are GPC3 negative. GPC3-1 and GPC3-2 constructs do not produce cytokine in the absence of target. Cytokine production appears to be antigen-density and affinity dependent. See FIGS. 4A and 4B.

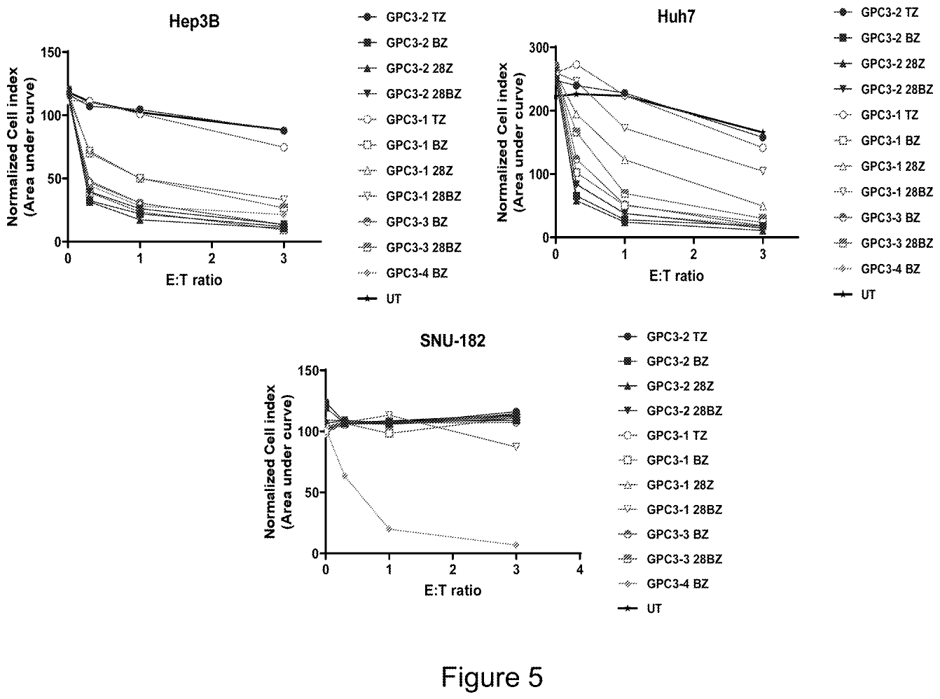

The GPC3-1 and GPC3-2 constructs were cytotoxic only to cells expressing GPC3, whereas the GPC3-4 construct was cytotoxic to both GPC3 positive cells (Hep3B and Huh7) and GPC3 negative cells (SNU-182). The lower affinity CAR (GPC3-1 BZ) displays equivalent cytotoxity to high affinity (GPC3-2 BZ) CAR. See FIG. 5.

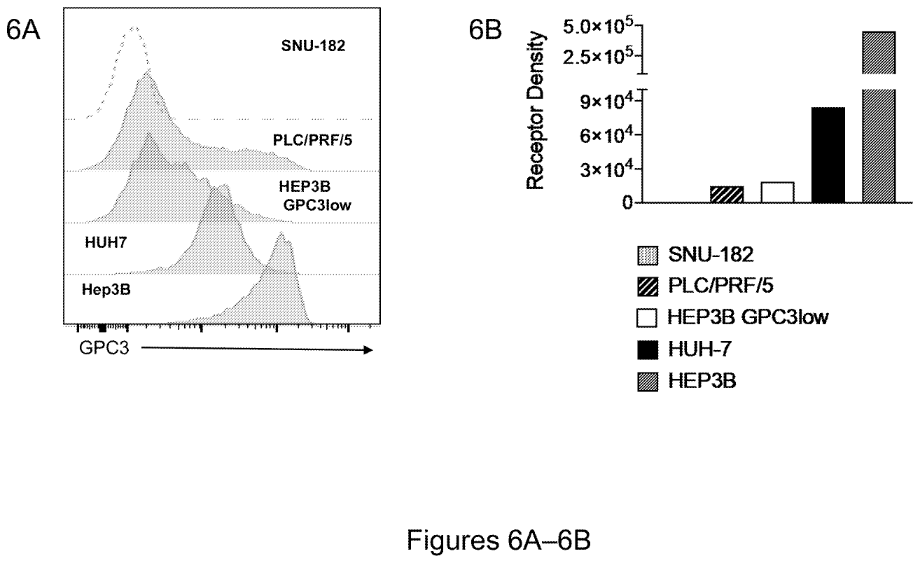

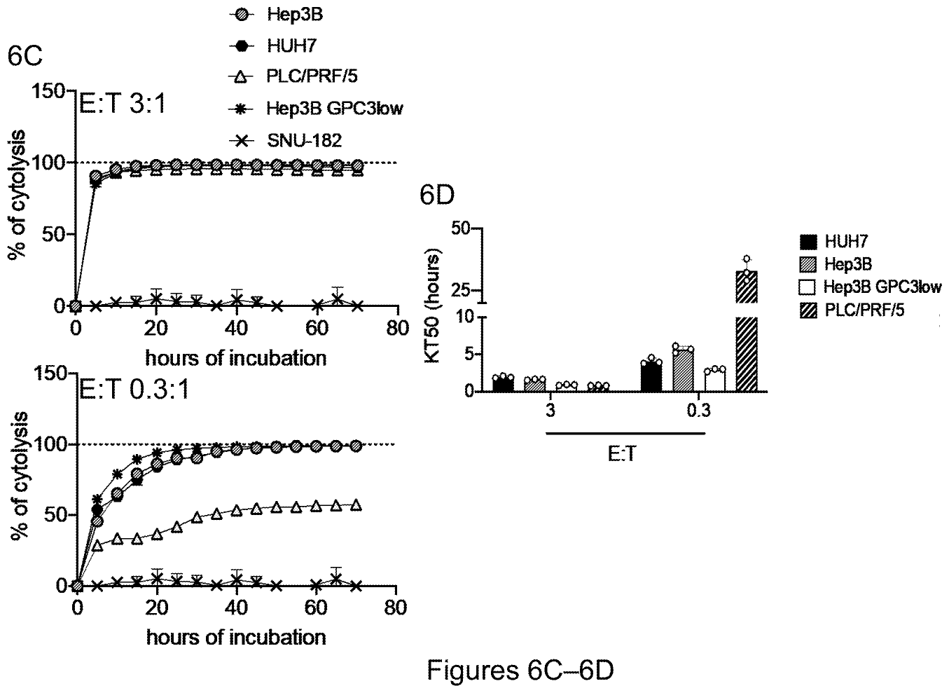

GPC3-1 BZ displayed cytotoxicity against HCC cell lines expressing low level of GPC3. All target cells tested were highly susceptible to killing by GPC3-1 at 3:1 and 0.3:1 E:T ratio, with only reduced killing seen in one of the cell lines with lower GPC3 expression at 0.3:1 E:T ratio. However, the completely comparable killing rate seen with our isogenic pair GPC3 high and low Hep3B cell line suggests that reduced antigen density is not a critical factor per se restricting CAR-T mediated cytolysis. See FIG. 6.

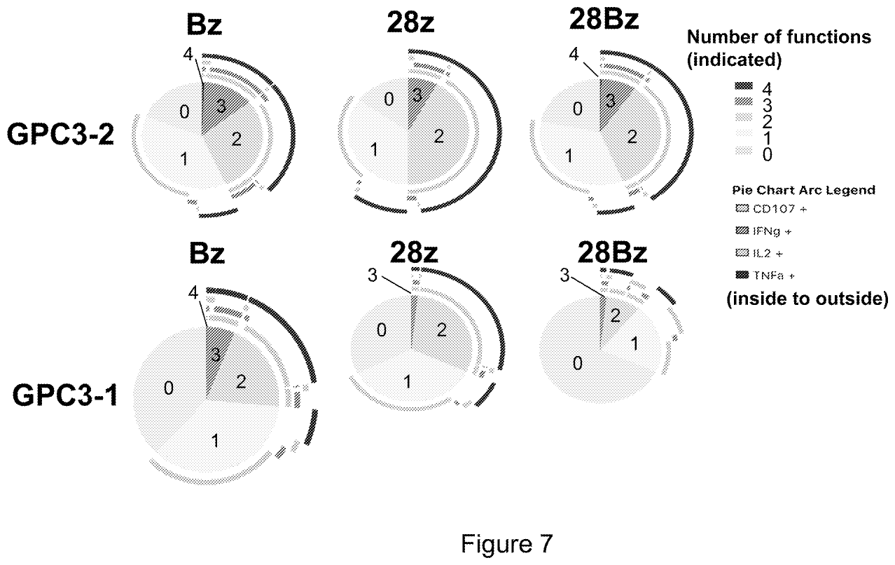

Both the GPC3-2 and GPC3-1 CAR T cells are polyfunctional irrespective of intracellular domain used. GPC3-1 BZ CAR T cells were polyfunctional, with a large proportion of cells displaying 2+ functions. Also, CAR T cells with CD28 were less polyfunctional in vitro than CAR T cells with the 4-1BB intracellular domain. See FIG. 7.

### Conclusion

Treatment with GPC3-1 resulted in the lowest overall cytokine production of the CARs tested. Both GPC3-1 and GPC3-2 were polyfunctional and specifically cytotoxic to cells expressing GPC3.

### Example 4: In Vivo Study of Multiple CAR Constructs in Hepatocellular Carcinoma Animal Model

### Summary

In the present example, anti-GPC3 CAR constructs were tested in vivo, and effects on body weight, tumors, and survival were compared.

### Methods

5×10⁶ Hep3B cells were implanted in the flanks of NSG mice (10 mice/group). When tumors reached an average volume of 150 mm³, mice were dosed with 4 million of GPC3-2 BZ or GPC3-1 BZ. Body weight, tumor volume (2×/week), and survival were monitored. Animals for which weight dropped to between 80 and 90 percent were given a food supplement; animals for which weight dropped below 80 percent were euthanized. Survival events (deaths) were determined by tumor size of greater than 1500 mm³. Each experiment was performed twice.

### Results

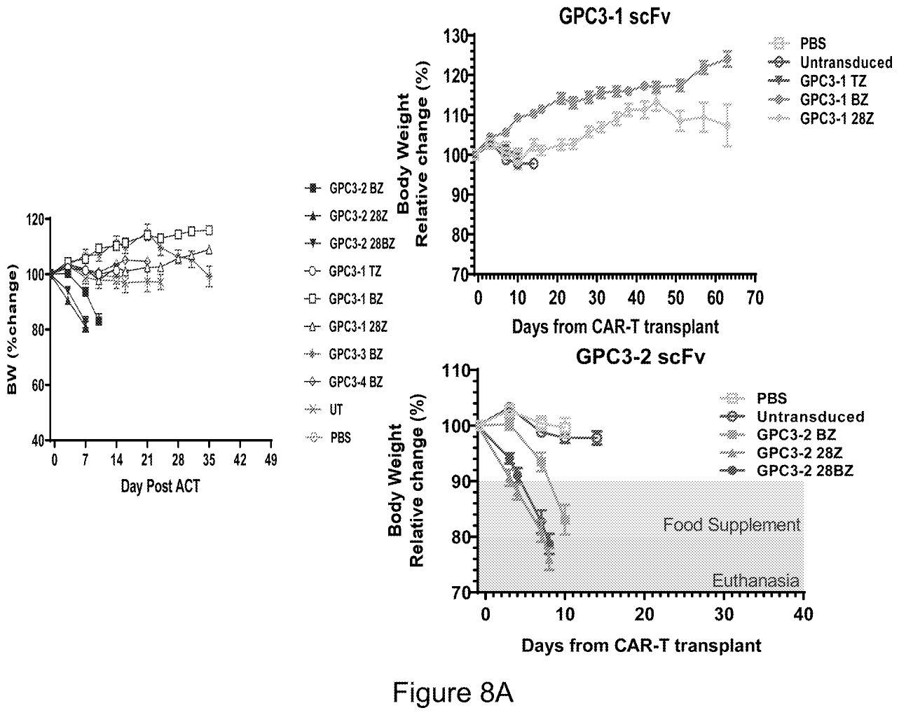

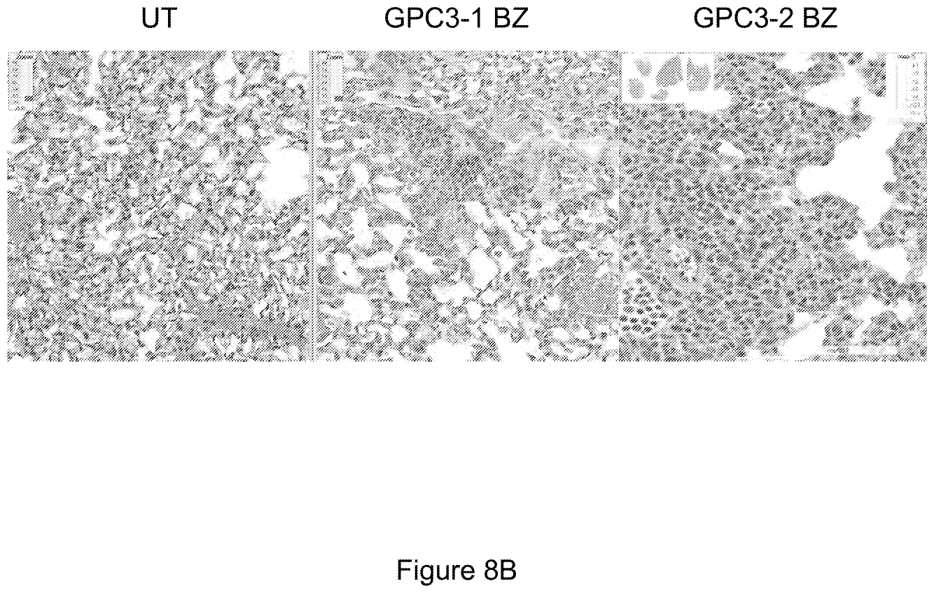

Body weight loss was observed with use of the high affinity GPC3-2 construct, but not the lower affinity GPC3-1 construct, indicating that the lower affinity binder is less toxic in vivo. GPC3-2 based CAR T cells were not tolerated at an equivalent in vivo dose to GPC3-1 based CAR T. The greater degree of toxicity of GPC3-2 BZ correlated with extensive infiltration of CAR T cells in normal mouse lung. Only a modest level of infiltrate was found in lungs of mice treated with GPC3-1 BZ. See FIGS. 8A and 8B.

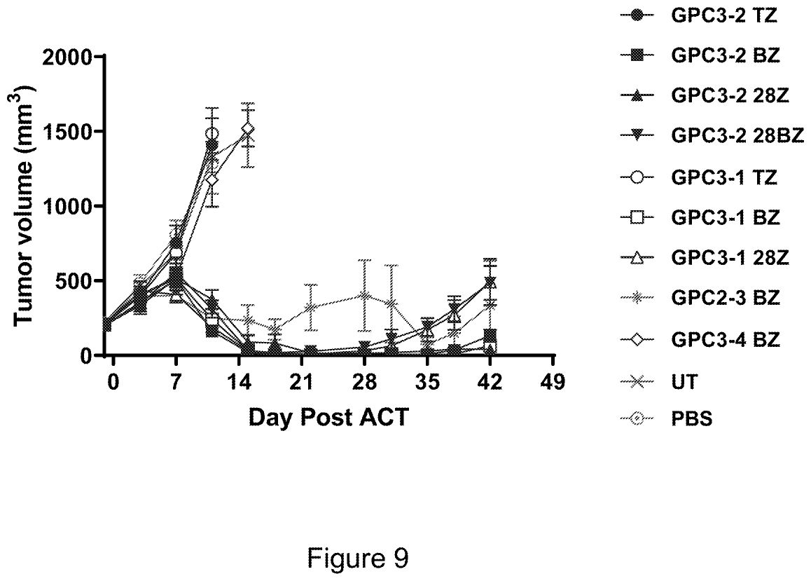

GPC3 CAR T induced Hep3B tumor regression in NSG mice. GPC3-1 BZ displayed superior anti-tumor activity over GPC3-3 BZ and GPC3-4 BZ. See FIG. 9.

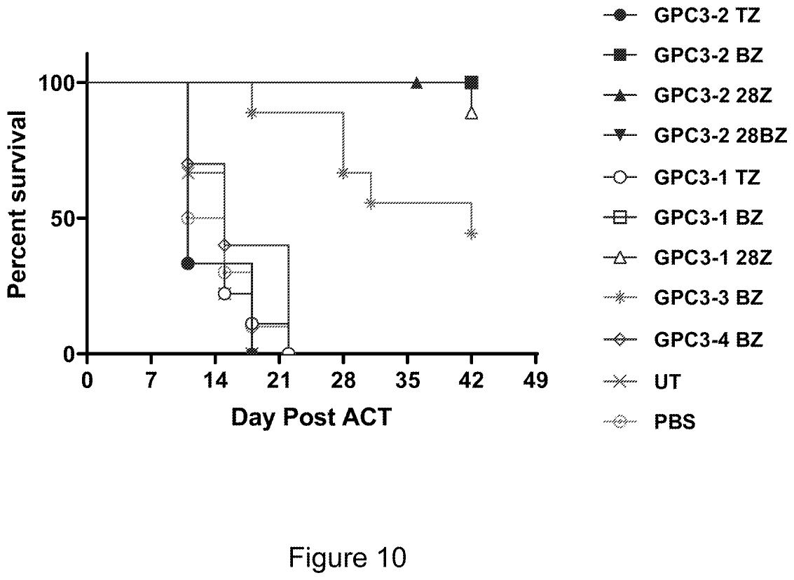

GPC3-1 and GPC3-2 CAR T prolonged survival in tumor-bearing NSG mice to a greater extent than either GPC3-3 or GPC3-4 CAR T, p<0.01 vs. GPC3-3 BZ; Kaplan-Meier w/Mantel Cox log-rank. See FIG. 10. Similarly, deletion of WPRE was determined to have no negative functional consequences on GPC3-1 BZ cells, in vitro or in vivo.

### Conclusion

Of the CARs tested, GPC3-1 BZ and GPC3-2 BZ displayed the most anti-tumor activity and provided the most survival benefits. GPC3-1 BZ displayed less toxicity and infiltration into normal tissue than did GPC3-2 BZ.

### Example 5: In Vivo Comparison of GPC3-1 CAR Constructs

### Summary

In the present example, multiple GPC3-1 CAR constructs comprising different signaling domains were tested in vivo and compared.

### Methods

Differentiation and Exhaustion analyses. Differentiation and exhaustion of multiple GPC3-1 CAR constructs were studied. Mice with Hep3B tumors were treated with CAR-T cells having different signaling domains (TZ=GPC3-1 TZ; BZ=GPC3-1 BZ; 28Z=GPC3-1 28Z), and spleen and tumors were analyzed 7 days after cell injection by flow cytometry. Differentiation and exhaustion was assayed using FACs detecting multiple markers in spleen and tumor cells. Differentiation status of T cells was analyzed by combined expression of CD62L and CD45RO (CD62L+/CD45RO-=naïve; CD62L+/CD45RO+=central memory; CD62L-/CD45RO+=effector memory; CD62L-/CD45RO-=effector memory cells re-expressing CD45RA (EMRA). CD3% was used as a measure of persistence and expansion.

Mice were injected with 5×10⁶ Hep3B cells to establish tumors of an average size of 150 mm³. Non tumor bearing mice or mice with Hep3B tumors were dosed with 4 million GPC3-1 BZ or GPC3-1 TZ T cells. Effect on body weight for both tumor bearing and non-bearing mice was measured up to 35 days after treatment. Tumor volume was also assayed twice a week. Animals were bled periodically after treatment for analysis of IFN-γ and TNF-α in blood. Cytokines were analyzed in the serum 8 days after CAR-T dosing. Each experiment was performed twice.

To investigate the potential for peripheral neurotoxicity in GPC3+ tumor-bearing (Hep3B HCC line) and non-tumor-bearing NSG mice, animals were administered human anti-GPC3 CAR-T cells. Histology was performed on tumor and intestinal nervous tissue of animals with Hep3B tumors treated with GPC3-1 BZ.

TABLE 3
Study Design

| | | | Day 1 Dose* | Necropsy^{a} |
|---|---|---|---|---|
| Group | Treatment | Tumor | (cells × 10⁶) | Day 15 |
| 1 | Vehicle Control | Y | 0 | 10F |
| 2 | GPC3-1 TZ** | Y | 5 | 10F |
| 3 | GPC3-1 BZ** | Y | 5 | 10F |
| 4 | Vehicle Control | N | 0 | 10F |
| 5 | GPC3-1 TZ** | N | 5 | 10F |
| 6 | GPC3-1 BZ | N | 5 | 10F |

^{a}Post peak CAR-T response but tumors present to harvest;
*Dose selected to completely regress Hep3B tumor;
**GPC3-1 TZ contains functional binding moiety without signaling domain;
**GPC3-1 BZ contains functional binding and signaling domain.

### Results

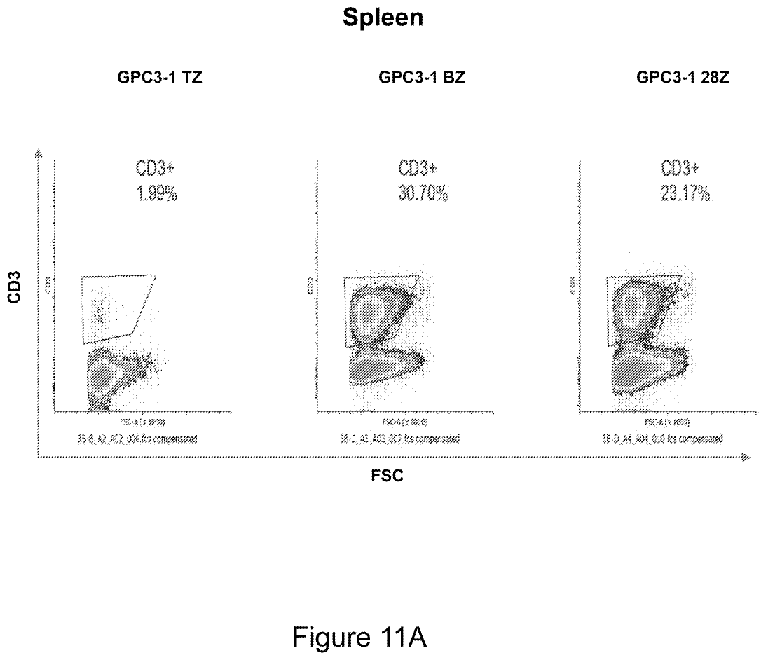

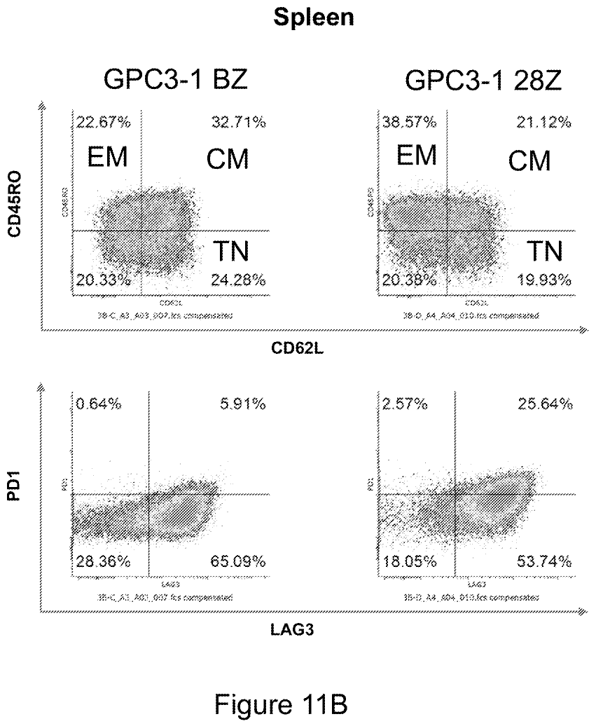

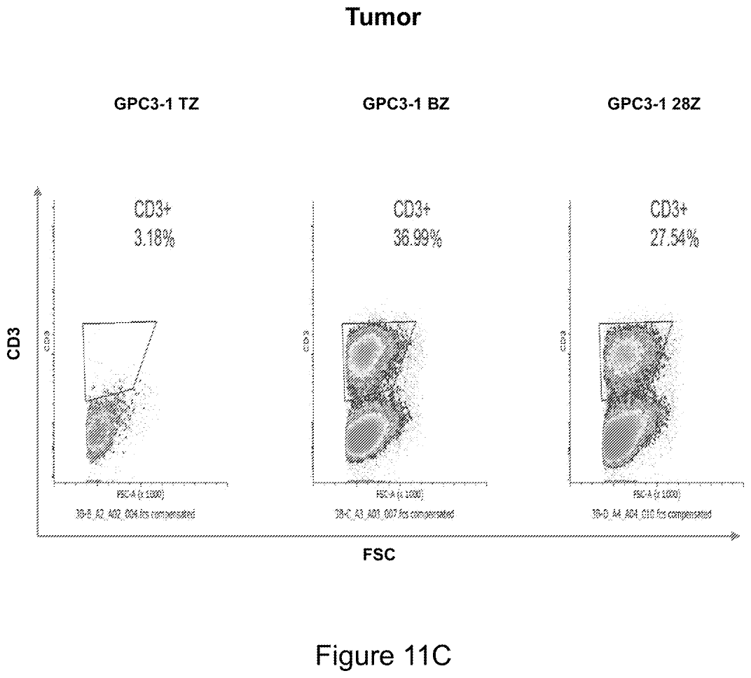

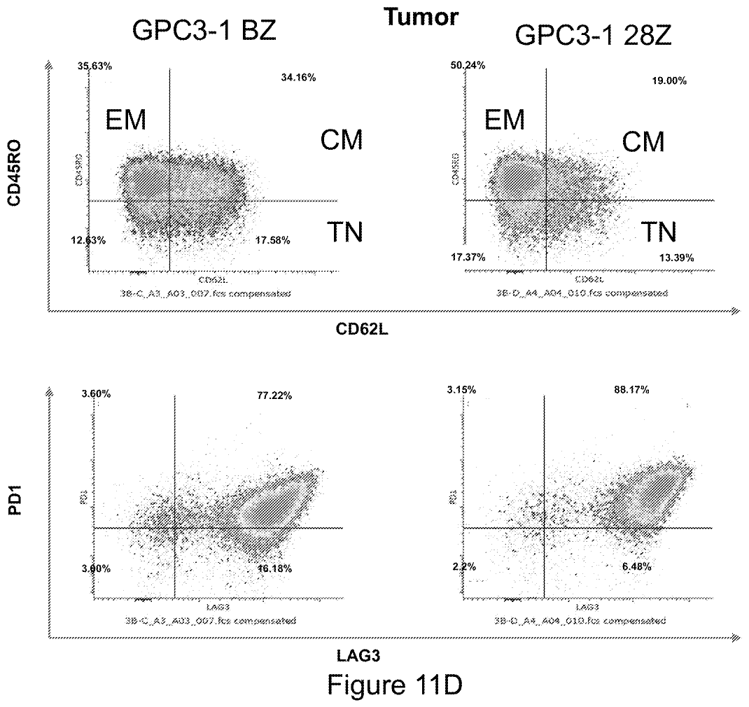

GPC3 CAR T with 4-1BB/CD3 zeta (BZ) signaling domains showed more central memory and less exhaustion than CD28/CD3 zeta (28Z) in vivo. Results show that GPC3-1 BZ CAR T cells in the spleen were less differentiated than GPC3-1 28Z CAR T cells, while retaining ability to fully activate and differentiate in the tumor, where the antigen is present. See FIGS. 11A-11D.

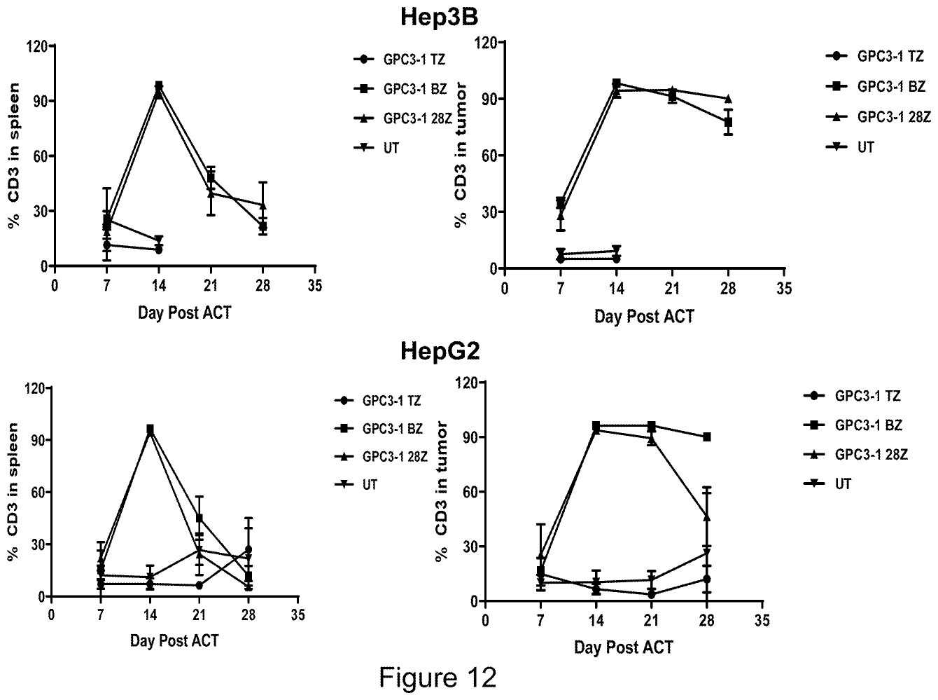

GPC3-1 BZ exhibited persistence. Expression of the activation/exhaustion markers LAG3 and PD1 confirmed that the GPC3-1 BZ CAR T cells maintained fewer activated/exhausted cells in the periphery. See FIG. 12.

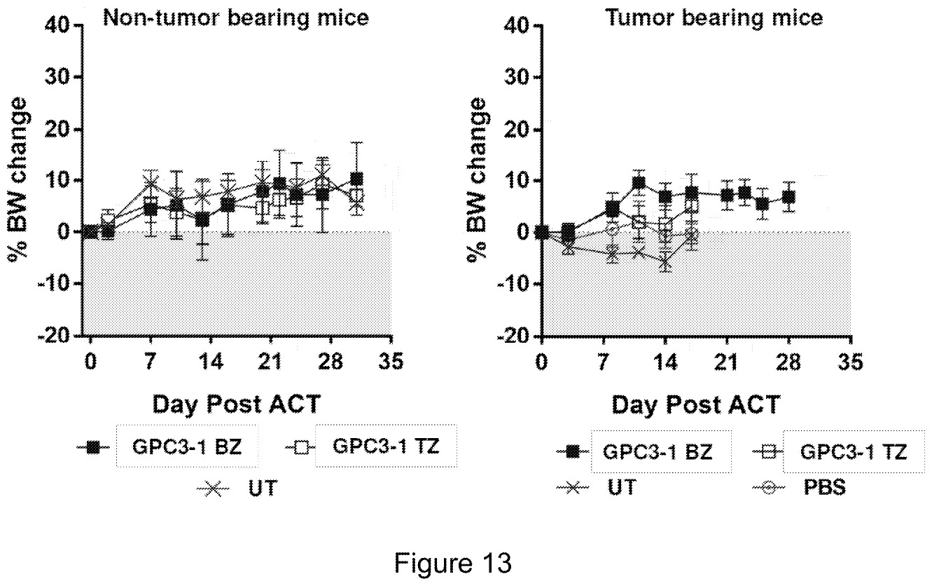

GPC3-1 BZ did not cause weight loss in either tumor or non-tumor bearing mice at tumor-regressing doses. See FIG. 13.

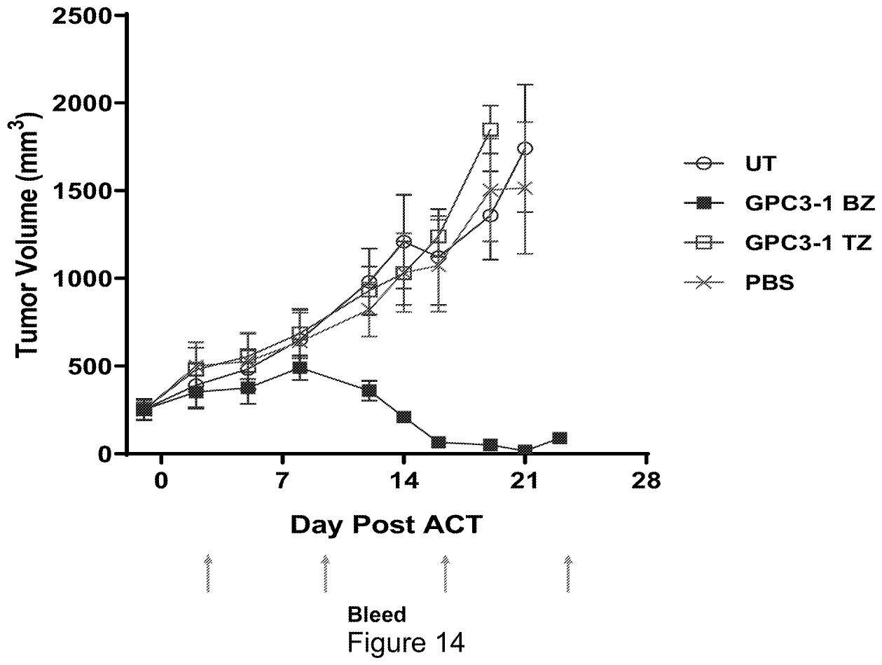

Complete tumor regression was observed only for GPC3-1 BZ CAR T cell treated mice. See FIG. 14.

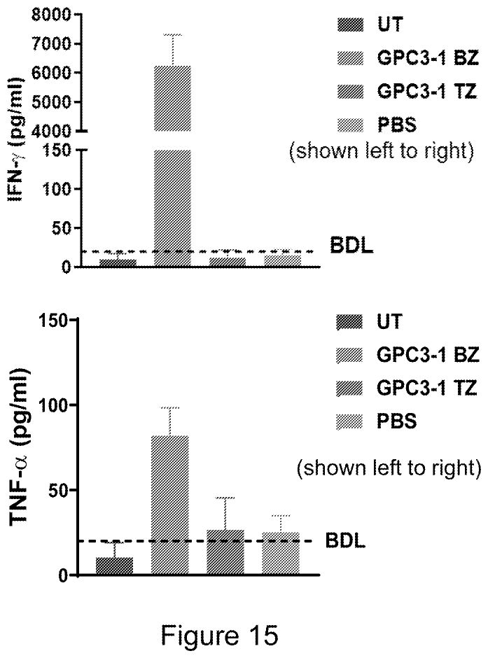

Minimal systemic cytokines (transient elevated IFN-y and TNF-a measured 7 days post infusion on day 8) were detected. Minimal and transient systemic cytokines were detected at efficacious CAR T doses and no weight loss was observed. Human IFN-γ and TNF-α were the only cytokines transiently detected at elevated levels in serum following regressive dose of CAR therapy. Levels of additional human or mouse cytokines including hIL-2, mIL-10, mIL-6, mTNFα, and mIFNγ were below the detectable limit (BDL). See FIG. 15.

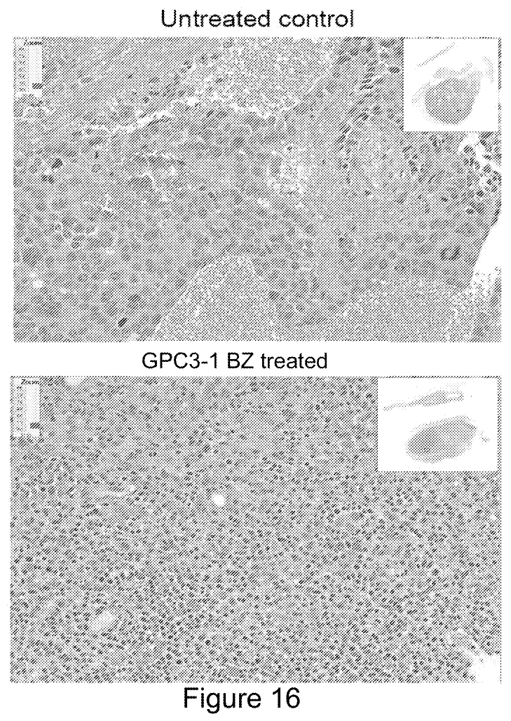

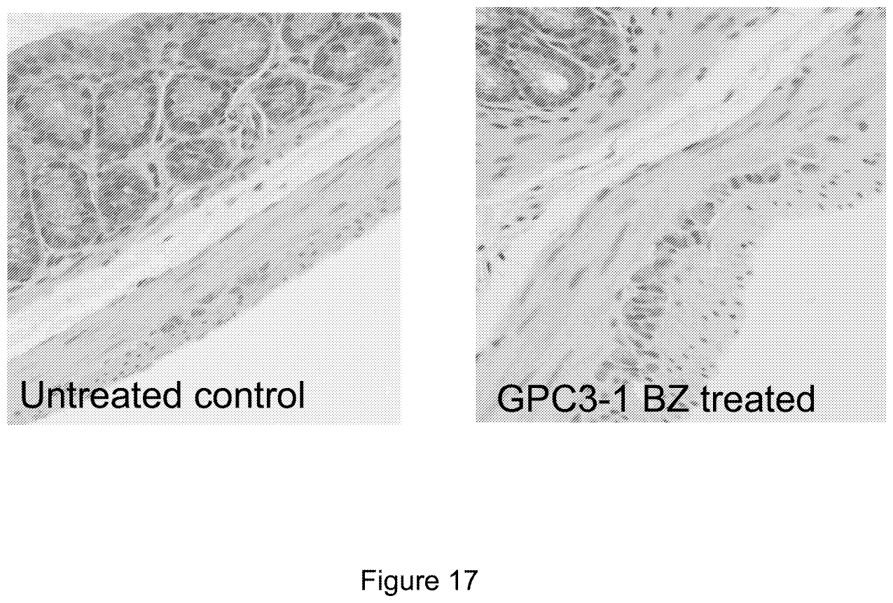

Tumor regression was accompanied by extensive T cell infiltration and expansion in the tumor. Tumors were smaller due to decreased neoplastic cells and were necrotic and infiltrated with mononuclear cells. The mice used lack lymphocytes; therefore, any mononuclear infiltrate is assumed to be human CAR-T cells. See FIG. 16. The intestinal nervous system, which expresses only low levels of GPC3, was unaffected. See FIG. 17. Minimal mononuclear infiltrates were observed in lung and liver (data not shown).

### Conclusion

As compared to constructs with other signaling domains, the GPC3-1 BZ construct is persistent, promotes a central memory response, and shows increased activity against tumors. Furthermore, treatment causes only a transient elevation of some cytokines and does not cause weight loss. Upon treatment, tumors are infiltrated with T cells and become necrotic, while normal tissue is unaffected.

### Example 6: Further Characterization of GPC3-1 BZ CAR T Cells

### Summary

In the present example, GPC3-1 BZ CAR T cells were further characterized in treatments of a variety of tumor types and cells with differing levels of GPC3 expression. Cytokine response was analyzed.

### Methods

Cytokine levels in response to GPC3-1 BZ CAR T cell treatments were investigated in tumor types with various levels of GPC3 expression. Immunohistochemistry was also performed on representative Hep3B and Huh7 tumor xenografts.

GPC3 expression analysis was also performed on cells within a tumor type. Staining intensity was graded on a scale of 1-4, with 1 being the lowest intensity and 4 being the highest intensity. A staining intensity of 2 indicates a low/moderate intensity. Relative expression of GPC3 expression was determined by FACs. GPC3 expression on Hep3B cells was determined by surface staining with fluorophore-labeled anti-GPC3 antibody and subsequent flow cytometric analysis. On the basis of GPC3 expression, Hep3B cells were gated as low, medium, or high expressers, and the frequency of GPC3 in each gate was plotted.

Cytokine levels were determined by ELISA. Cell lines were co-cultured with GPC3-1 BZ CAR T cells at a 1:1 ratio in RPMI 10% FCS. After 24 hours, supernatants were collected, and cytokines analyzed by Meso Scale Discovery 4-plex Kit to detect IFN-γ, IL-2, TNF-α, and IL-10. Cells were exposed to GPC3-1 BZ T cells for 24 hours before cytokine analysis. Cytokine levels in different cell types were tested after GPC3-1 BZ treatment.

### Results

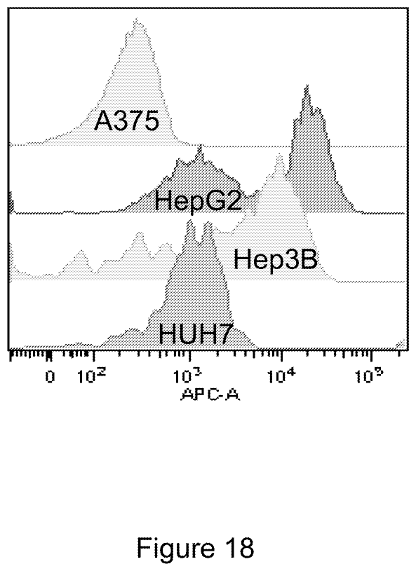

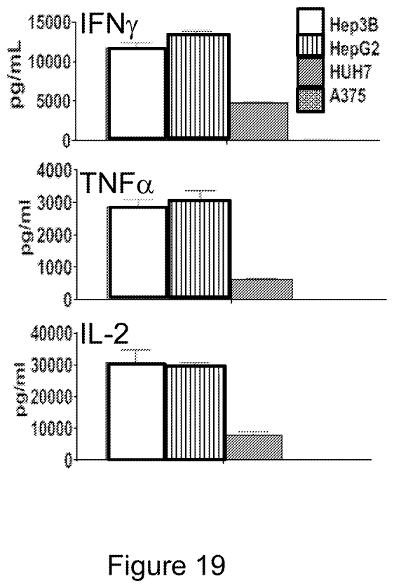

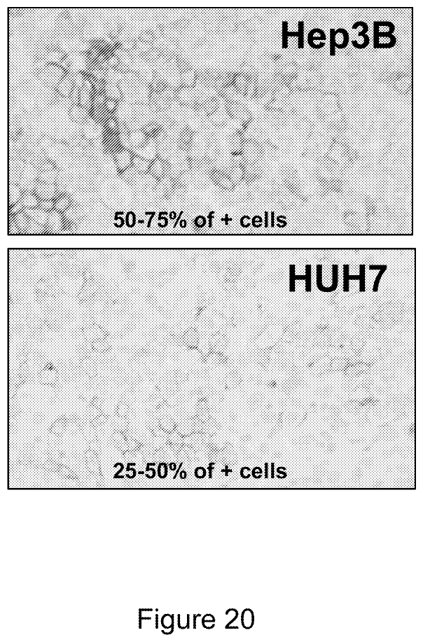

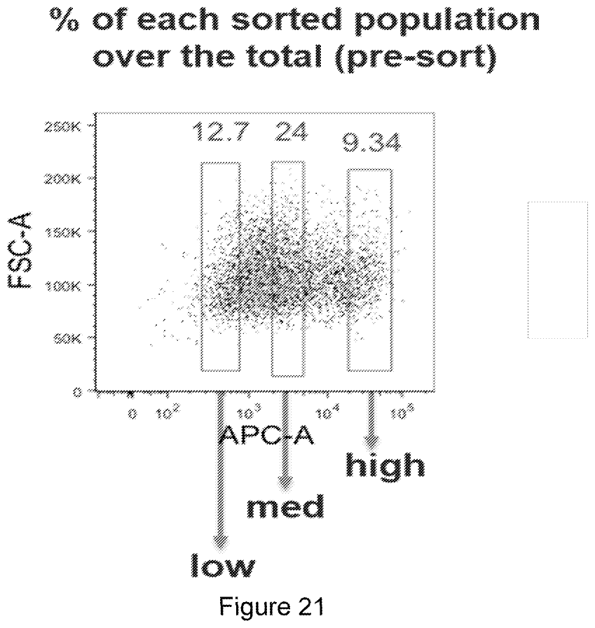

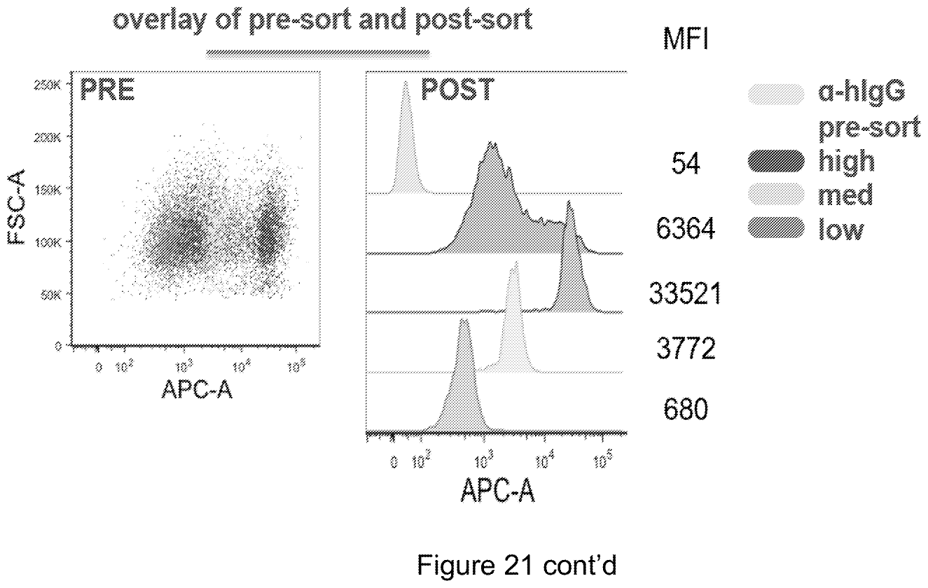

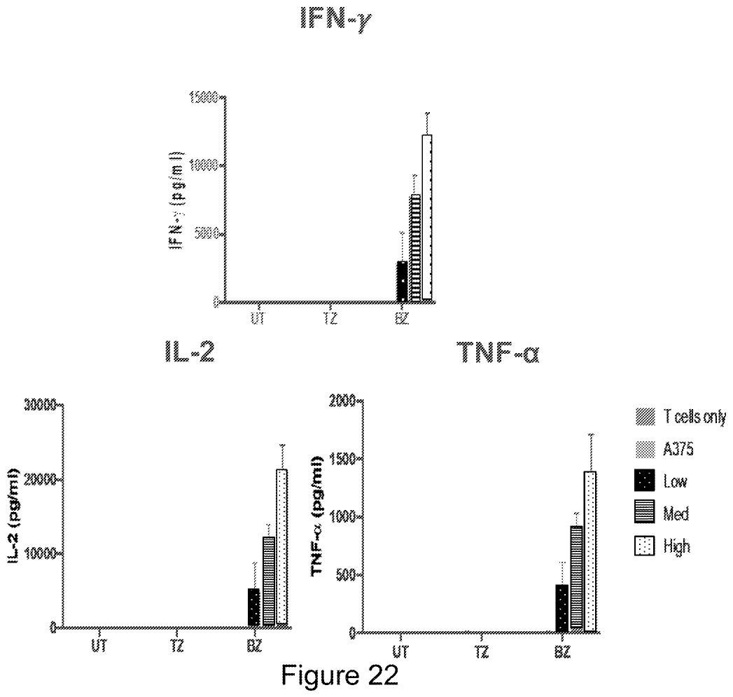

GPC3-1 BZ CAR T cell-induced cytokine output in GPC3 expressing cell lines was proportional to surface GPC3 expression. See FIGS. 18-22.

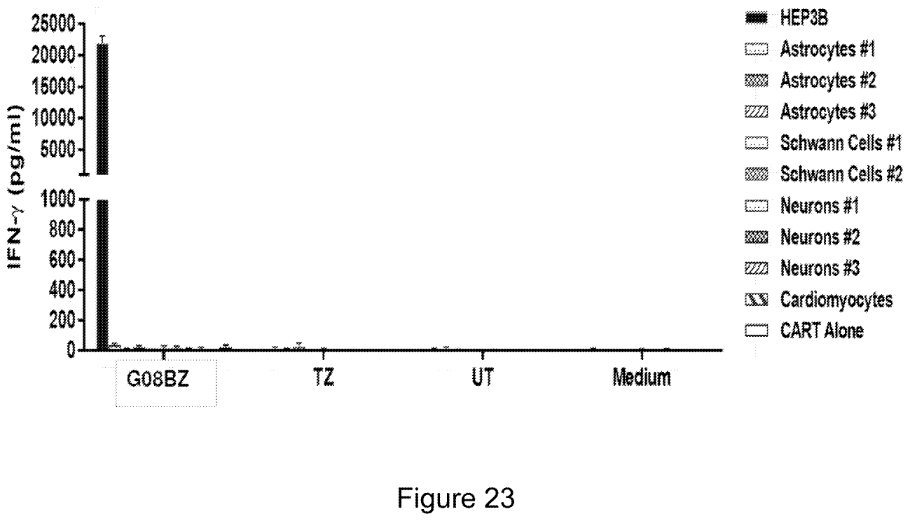

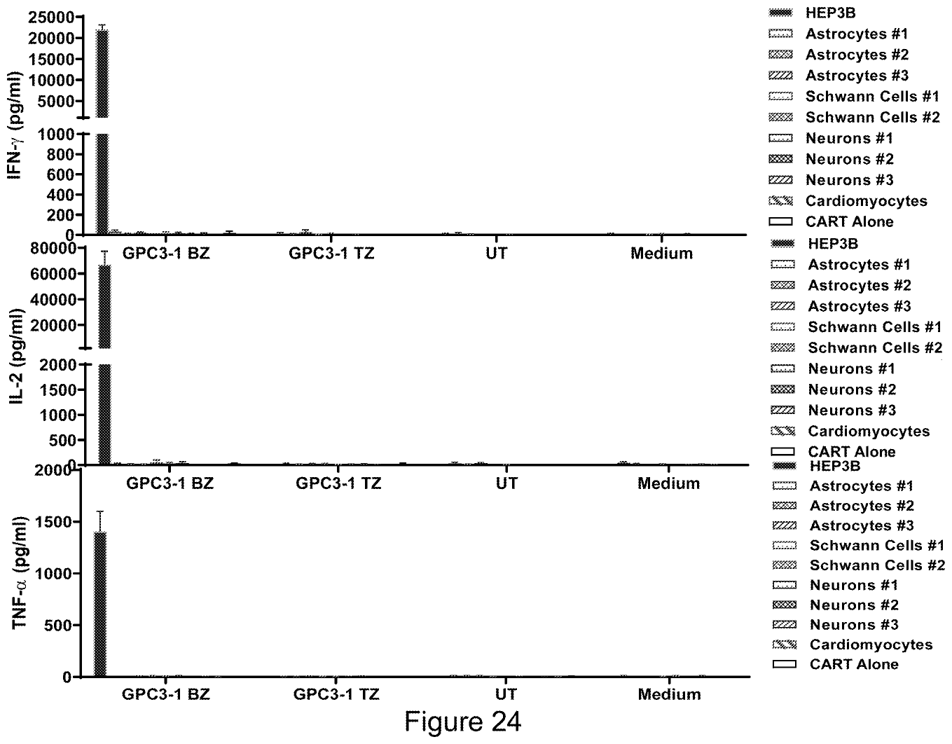

GPC3-1 BZ CAR T cells did not cause a cytokine response in GPC3-negative or normal tissue. See FIGS. 23 and 24.

### Conclusion

GPC3-1 BZ CAR T cells induce cytokine output at levels proportional to the GPC3 expression of the treated cells.

### Example 7: Treatment with GPC3-1 CAR T Cell Constructs and Anti-Cytokine Antibodies

### Summary

In the present example, GPC3-1 CAR T cell therapy was attempted in conjunction with anti-cytokine antibodies.

### Methods

Tumors were treated with different combinations of GPC3-1 CAR T cell therapy and anti-cytokine antibodies. Mice with Hep3B tumors (10 mice/group) were treated with 5 million GPC3-1 BZ or GPC3-1 TZ transduced cells (TZ=truncated CD3 zeta, a non-signaling negative control), in the presence or absence of 100 μg anti-human TNFα (golimumab, Janssen) or anti-mouse IL-6 (Bio X Cell).

A resistant model of HCC, Huh7, was used to test high doses (1e7-3e7) of GPC3-1 CAR T in conjunction with two different timings of anti-TNF-α administration. Mice with Huh7 tumors (10 mice/group) were treated with the indicated dose of GPC3-1 BZ T cells (10 or 30 million cells), and 100 μg anti-TNFα was dosed on the same day as the CAR T treatment (Day 0), or two days after initiating treatment (Day 2).

### Results

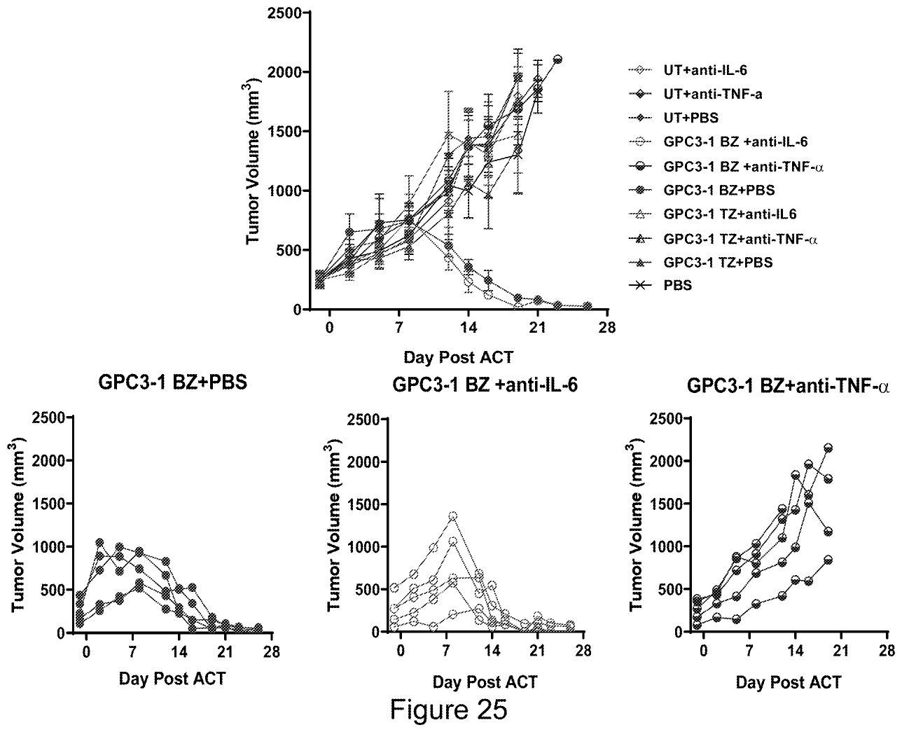

Blocking TNF-α but not IL-6 abolished the efficacy from GPC3-1 BZ. See FIG. 25.

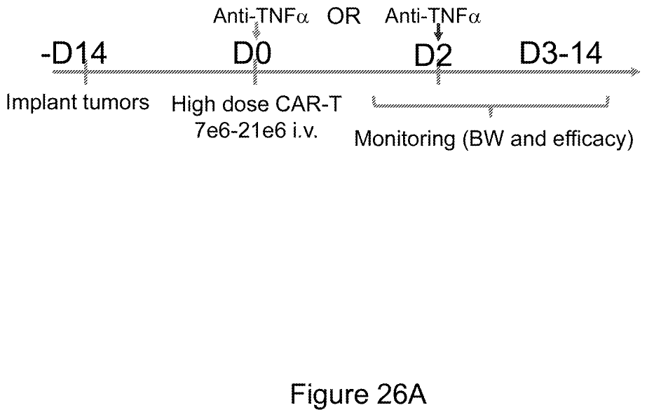

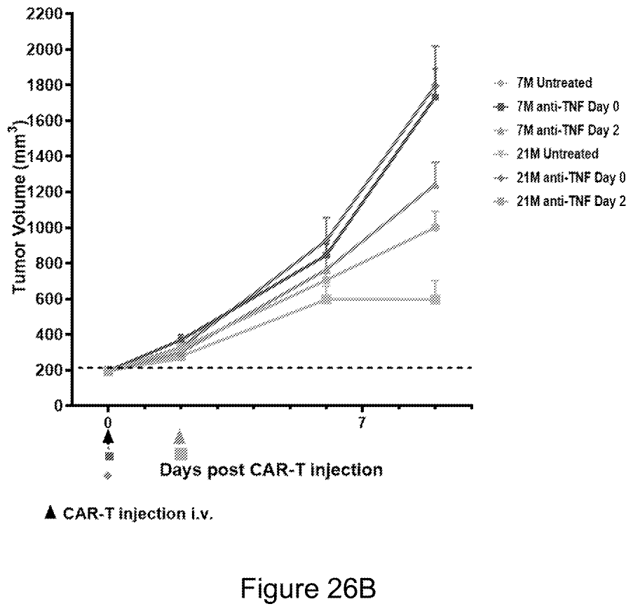

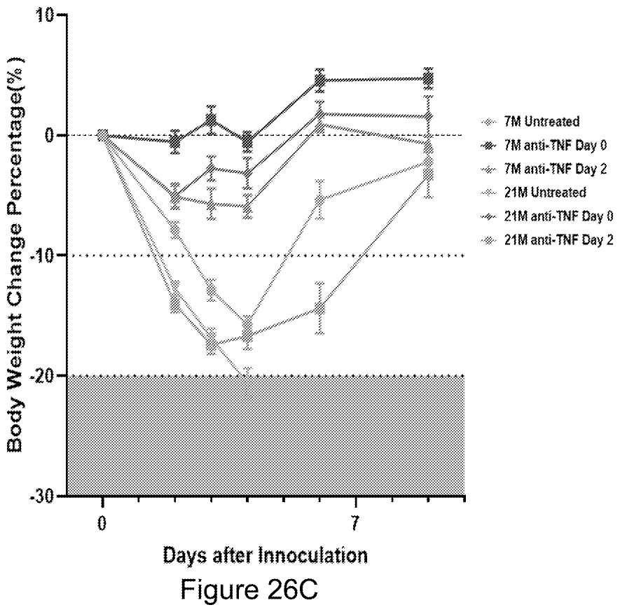

Higher CAR T cell doses are needed to cause tumor growth inhibition in resistant HCC model Huh7, but higher doses are also associated with CRS like symptoms and weight loss. Weight loss was reversed to achieve tumor growth inhibition with anti-TNFα using delayed dosing. See FIGS. 26A-26C.

### Conclusion

Use of anti-TNFα treatment in conjunction with GPC3-1 BZ therapy can mitigate the weight loss effects of high dosage CAR T cell therapy.

The embodiments described herein can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present invention.

TABLE 3
Sequences used in the Examples.

| | |
|---|---|
| SEQ ID NO: 1 | GPC3-1 scFv amino acid sequence |
| SEQ ID NO: 2 | GPC3-2 scFv amino acid sequence |
| SEQ ID NO: 3 | GPC3-1 BZ CAR amino acid sequence |
| SEQ ID NO: 4 | GPC3-1 TZ CAR amino acid sequence |
| SEQ ID NO: 5 | GPC3-1 28Z CAR amino acid sequence |
| SEQ ID NO: 6 | GPC3-1 28 BZ CAR amino acid sequence |
| SEQ ID NO: 7 | GPC3-2 BZ CAR amino acid sequence |
| SEQ ID NO: 8 | GPC3-2 TZ CAR amino acid sequence |
| SEQ ID NO: 9 | GPC3-2 28Z CAR amino acid sequence |
| SEQ ID NO: 10 | GPC3-2 28BZ CAR amino acid sequence |
| SEQ ID NO: 11 | GPC3-1 BZ CAR nucleic acid sequence |
| SEQ ID NO: 12 | GPC3-1 TZ CAR nucleic acid sequence |
| SEQ ID NO: 13 | GPC3-1 28Z CAR nucleic acid sequence |
| SEQ ID NO: 14 | GPC3-1 28BZ CAR nucleic acid sequence |
| SEQ ID NO: 15 | GPC3-2 BZ CAR nucleic acid sequence |
| SEQ ID NO: 16 | GPC3-2 TZ CAR nucleic acid sequence |
| SEQ ID NO: 17 | GPC3-2 28Z CAR nucleic acid sequence |
| SEQ ID NO: 18 | GPC3-2 28BZ CAR nucleic acid sequence |
| SEQ ID NO: 19 | GPC3-3 BZ CAR amino acid sequence |
| SEQ ID NO: 20 | GPC3-3 28BZ CAR amino acid sequence |
| SEQ ID NO: 21 | GPC3-4 BZ CAR amino acid sequence |
| SEQ ID NO: 22 | GPC3-3 BZ CAR nucleic acid sequence |
| SEQ ID NO: 23 | GPC3-3 28BZ CAR nucleic acid sequence |
| SEQ ID NO: 24 | GPC3-4 CAR nucleic acid sequence |
| SEQ ID NO: 25 | GPC3-1 BZ CAR amino acid sequence |
| | (WPRE-deleted) |
| SEQ ID NO: 26 | GPC3-1 BZ CAR nucleic acid sequence |
| | (WPRE-deleted) |
| SEQ ID NO: 27 | GPC3-1 VH |
| SEQ ID NO: 28 | GPC3-1 VL |
| SEQ ID NO: 29 | GPC3-2 VH |
| SEQ ID NO: 30 | GPC3-2 VL |
| SEQ ID NO: 31 | GPC3-3 scFv amino acid sequence |
| SEQ ID NO: 32 | GPC3-4 scFv amino acid sequence |
| SEQ ID NO: 33 | GPC3-1 scFv nucleic acid sequence |
| SEQ ID NO: 34 | GPC3-2 scFv nucleic acid sequence |
| SEQ ID NO: 35 | GPC3-3 scFv nucleic acid sequence |
| SEQ ID NO: 36 | GPC3-4 scFv nucleic acid sequence |
| SEQ ID NO: 37 | GPC3-1 and GPC3-2 VH CDR1 |
| SEQ ID NO: 38 | GPC3-1 and GPC3-2 VH CDR2 |
| SEQ ID NO: 39 | GPC3-1 and GPC3-2 VH CDR3 |
| SEQ ID NO: 40 | GPC3-1 VL CDR1 |
| SEQ ID NO: 41 | GPC3-1 VL CDR2 |
| SEQ ID NO: 42 | GPC3-1 VL CDR3 |
| SEQ ID NO: 43 | GPC3-2 VL CDR1 |
| SEQ ID NO: 44 | GPC3-2 VL CDR2 |
| SEQ ID NO: 45 | GPC3-2 VL CDR3 |

TABLE 4
Sequences

| | |
|---|---|
| SEQ ID NO: 1 | EVQLLESGGGLVQPGGSLRLSCAASGFTFSSYAMSWVRQAPG |
| | KGLEWVSAISGSGGSTYYADSVKGRFTISRDNSKNTLYLQMN |
| | SLRAEDTAVYYCARGKRYFDYWGQGTMVTVSSGGGGSGGG |
| | GSGGGGSSYELTQPPSASGTPGQRVTISCSGGSSNIGSNTVNW |
| | FRQLPGTAPKLLVYFNNQRPSGVPDRFSGSKSGTSASLAIGGL |
| | QSDDEADYYCVAWDDSLNAPVFGGGTKVTVL |
| SEQ ID NO: 2 | EVQLLESGGGLVQPGGSLRLSCAASGFTFSSYAMSWVRQAPG |
| | KGLEWVSAISGSGGSTYYADSVKGRFTISRDNSKNTLYLQMN |
| | SLRAEDTAVYYCAKGKRYFDYWGQGTMVTVSSGGGGSGGG |
| | GSGGGGSQSVLTQPPSASGTPGQRVTISCSGGSSDIGSNTVNW |
| | YQQLPGTAPKLLIYYNNQRPSGVPDRFSGSKSGTSASLAISGL |
| | QSEDEADYYCATWDDRMYSPVFGGGTKLTVL |
| SEQ ID NO: 3 | MLLLVTSLLLCELPHPAFLLIPGVHSEVQLLESGGGLVQPGGS |
| | LRLSCAASGFTFSSYAMSWVRQAPGKGLEWVSAISGSGGSTY |
| | YADSVKGRFTISRDNSKNTLYLQMNSLRAEDTAVYYCARGK |
| | RYFDYWGQGTMVTVSSGGGGSGGGGSGGGGSSYELTQPPSA |
| | SGTPGQRVTISCSGGSSNIGSNTVNWFRQLPGTAPKLLVYFNN |
| | QRPSGVPDRFSGSKSGTSASLAIGGLQSDDEADYYCVAWDDS |
| | LNAPVFGGGTKVTVLESKYGPPCPPCPFWVLVVVGGVLACYS |
| | LLVTVAFIIFWVKRGRKKLLYIFKQPFMRPVQTTQEEDGCSCR |
| | FPEEEEGGCELRVKFSRSADAPAYQQGQNQLYNELNLGRREE |
| | YDVLDKRRGRDPEMGGKPRRKNPQEGLYNELQKDKMAEAY |
| | SEIGMKGERRRGKGHDGLYQGLSTATKDTYDALHMQALPPR |
| | * |
| SEQ ID NO: 4 | MLLLVTSLLLCELPHPAFLLIPGVHSEVQLLESGGGLVQPGGS |
| | LRLSCAASGFTFSSYAMSWVRQAPGKGLEWVSAISGSGGSTY |
| | YADSVKGRFTISRDNSKNTLYLQMNSLRAEDTAVYYCARGK |
| | RYFDYWGQGTMVTVSSGGGGSGGGGSGGGGSSYELTQPPSA |
| | SGTPGQRVTISCSGGSSNIGSNTVNWFRQLPGTAPKLLVYFNN |
| | QRPSGVPDRFSGSKSGTSASLAIGGLQSDDEADYYCVAWDDS |
| | LNAPVFGGGTKVTVLESKYGPPCPPCPFWVLVVVGGVLACYS |
| | LLVTVAFIIFWVRVKFSRSADAPA* |
| SEQ ID NO: 5 | MLLLVTSLLLCELPHPAFLLIPGVHSEVQLLESGGGLVQPGGS |
| | LRLSCAASGFTFSSYAMSWVRQAPGKGLEWVSAISGSGGSTY |
| | YADSVKGRFTISRDNSKNTLYLQMNSLRAEDTAVYYCARGK |
| | RYFDYWGQGTMVTVSSGGGGSGGGGSGGGGSSYELTQPPSA |
| | SGTPGQRVTISCSGGSSNIGSNTVNWFRQLPGTAPKLLVYFNN |
| | QRPSGVPDRFSGSKSGTSASLAIGGLQSDDEADYYCVAWDDS |
| | LNAPVFGGGTKVTVLESKYGPPCPPCPFWVLVVVGGVLACYS |
| | LLVTVAFIIFWVRSKRSRLLHSDYMNMTPRRPGPTRKHYQPY |
| | APPRDFAAYRSRVKFSRSADAPAYQQGQNQLYNELNLGRRE |
| | EYDVLDKRRGRDPEMGGKPRRKNPQEGLYNELQKDKMAEA |
| | YSEIGMKGERRRGKGHDGLYQGLSTATKDTYDALHMQALPP |
| | R* |
| SEQ ID NO: 6 | MLLLVTSLLLCELPHPAFLLIPGVHSEVQLLESGGGLVQPGGS |
| | LRLSCAASGFTFSSYAMSWVRQAPGKGLEWVSAISGSGGSTY |
| | YADSVKGRFTISRDNSKNTLYLQMNSLRAEDTAVYYCARGK |
| | RYFDYWGQGTMVTVSSGGGGSGGGGSGGGGSSYELTQPPSA |
| | SGTPGQRVTISCSGGSSNIGSNTVNWFRQLPGTAPKLLVYFNN |
| | QRPSGVPDRFSGSKSGTSASLAIGGLQSDDEADYYCVAWDDS |
| | LNAPVFGGGTKVTVLESKYGPPCPPCPFWVLVVVGGVLACYS |
| | LLVTVAFIIFWVRSKRSRLLHSDYMNMTPRRPGPTRKHYQPY |
| | APPRDFAAYRSKRGRKKLLYIFKQPFMRPVQTTQEEDGCSCR |
| | FPEEEEGGCELRVKFSRSADAPAYQQGQNQLYNELNLGRREE |
| | YDVLDKRRGRDPEMGGKPRRKNPQEGLYNELQKDKMAEAY |
| | SEIGMKGERRRGKGHDGLYQGLSTATKDTYDALHMQALPPR |
| | * |
| SEQ ID NO: 7 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCAKGKRYFD |
| | YWGQGTMVTVSSGGGGSGGGGSGGGGSQSVLTQPPSASGTP |
| | GQRVTISCSGGSSDIGSNTVNWYQQLPGTAPKLLIYYNNQRPS |
| | GVPDRFSGSKSGTSASLAISGLQSEDEADYYCATWDDRMYSP |
| | VFGGGTKLTVLESKYGPPCPPCPFWVLVVVGGVLACYSLLVT |
| | VAFIIFWVKRGRKKLLYIFKQPFMRPVQTTQEEDGCSCRFPEE |
| | EEGGCELRVKFSRSADAPAYQQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR* |
| SEQ ID NO: 8 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCAKGKRYFD |
| | YWGQGTMVTVSSGGGGSGGGGSGGGGSQSVLTQPPSASGTP |
| | GQRVTISCSGGSSDIGSNTVNWYQQLPGTAPKLLIYYNNQRPS |
| | GVPDRFSGSKSGTSASLAISGLQSEDEADYYCATWDDRMYSP |
| | VFGGGTKLTVLESKYGPPCPPCPFWVLVVVGGVLACYSLLVT |
| | VAFIIFWVRVKFSRSADAPA* |
| SEQ ID NO: 9 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCAKGKRYFD |
| | YWGQGTMVTVSSGGGGSGGGGSGGGGSQSVLTQPPSASGTP |
| | GQRVTISCSGGSSDIGSNTVNWYQQLPGTAPKLLIYYNNQRPS |
| | GVPDRFSGSKSGTSASLAISGLQSEDEADYYCATWDDRMYSP |
| | VFGGGTKLTVLESKYGPPCPPCPFWVLVVVGGVLACYSLLVT |
| | VAFIIFWVRSKRSRLLHSDYMNMTPRRPGPTRKHYQPYAPPR |
| | DFAAYRSRVKFSRSADAPAYQQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR* |
| SEQ ID NO: 10 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCAKGKRYFD |
| | YWGQGTMVTVSSGGGGSGGGGSGGGGSQSVLTQPPSASGTP |
| | GQRVTISCSGGSSDIGSNTVNWYQQLPGTAPKLLIYYNNQRPS |
| | GVPDRFSGSKSGTSASLAISGLQSEDEADYYCATWDDRMYSP |
| | VFGGGTKLTVLESKYGPPCPPCPFWVLVVVGGVLACYSLLVT |
| | VAFIIFWVRSKRSRLLHSDYMNMTPRRPGPTRKHYQPYAPPR |
| | DFAAYRSKRGRKKLLYIFKQPFMRPVQTTQEEDGCSCRFPEEE |
| | EGGCELRVKFSRSADAPAYQQGQNQLYNELNLGRREEYDVL |
| | DKRRGRDPEMGGKPRRKNPQEGLYNELQKDKMAEAYSEIG |
| | MKGERRRGKGHDGLYQGLSTATKDTYDALHMQALPPR* |
| SEQ ID NO: 11 | atgctgctgctggtgacaagcctgctgctgtgcgaactgccccatcccgccttcctgctgattcctg |
| | gtgtacactccgaggtgcagctgttggagtctgggggaggcttggtacagcctggggggtccctg |
| | agactctcctgtgcagcctctggattcacctttagcagctatgccatgagctgggtccgccaggctc |
| | cagggaaggggctggagtgggtctcagctattagtggtagtggtggtagcacatactacgcagac |
| | tccgtgaagggccggttcaccatctccagagacaattccaagaacacgctgtatctgcaaatgaac |
| | agcctgagagccgaggacacggccgtgtattactgtgcgagaggaaagcgatactttgactactg |
| | gggccaggggacaatggtcaccgtctcgagtggtggggggggcagcggtggtggaggctctg |
| | gtggaggagggagctcctatgagctgactcagccaccctcagcgtctgggacccccgggcaga |
| | gggtcaccatctcttgttctggaggcagctccaacatcggaagtaatactgtaaactggttccggca |
| | gctcccaggaacggcccccaaactcctcgtttattttaataatcagcgaccctcaggggtccctga |
| | ccgattctctggctccaagtctggcacctcggcctccctggccatcggtgggctccagtctgacga |
| | tgaggctgactattactgtgtagcatgggatgactctctgaatgctccggtgttcggcggagggac |
| | caaggtcaccgtcctagagagcaaatatggaccaccatgccctccatgtcctttttgggtcctggtg |
| | gtcgtgggaggcgtgctggcatgttattctctgctggtcacagtggctttcatcatcttctgggtcaa |
| | gcgaggccggaagaaactgctgtacatcttcaaacagccttttatgcgcccagtgcagacaactc |
| | aggaggaagacggctgctcttgtcggttccccgaggaagaggaagggggatgtgagctgcgcg |
| | tgaagttttctcgaagtgccgatgctcctgcatatcagcagggacagaaccagctgtacaacgagc |
| | tgaatctgggccggagagaggaatacgacgtgctggataagaggcgcggcagagacccagaa |
| | atgggcgggaagccacgacggaaaaacccccaggaggggctgtataatgaactgcagaagga |
| | caaaatggccgaggcttacagcgaaatcgggatgaagggagagagaaggcgcggaaaaggc |
| | cacgatggactgtatcagggcctgagcactgccaccaaggacacctacgatgctctgcacatgca |
| | ggcactgccacccaggtga |
| SEQ ID NO: 12 | atgctgctgctggtgacaagcctgctgctgtgcgaactgccccatcccgccttcctgctgattcctg |
| | gtgtacactccgaggtgcagctgttggagtctgggggaggcttggtacagcctggggggtccctg |
| | agactctcctgtgcagcctctggattcacctttagcagctatgccatgagctgggtccgccaggctc |
| | cagggaaggggctggagtgggtctcagctattagtggtagtggtggtagcacatactacgcagac |
| | tccgtgaagggccggttcaccatctccagagacaattccaagaacacgctgtatctgcaaatgaac |
| | agcctgagagccgaggacacggccgtgtattactgtgcgagaggaaagcgatactttgactactg |
| | gggccaggggacaatggtcaccgtctcgagtggtggggggggcagcggtggtggaggctctg |
| | gtggaggagggagctcctatgagctgactcagccaccctcagcgtctgggacccccgggcaga |
| | gggtcaccatctcttgttctggaggcagctccaacatcggaagtaatactgtaaactggttccggca |
| | gctcccaggaacggcccccaaactcctcgtttattttaataatcagcgaccctcaggggtccctga |
| | ccgattctctggctccaagtctggcacctcggcctccctggccatcggtgggctccagtctgacga |
| | tgaggctgactattactgtgtagcatgggatgactctctgaatgctccggtgttcggcggagggac |
| | caaggtcaccgtcctagagagcaaatatggaccaccatgccctccatgtcctttttgggtcctggtg |
| | gtcgtgggaggcgtgctggcatgttattctctgctggtcacagtggctttcatcatcttctgggtccg |
| | cgtgaagttttctcgaagtgccgatgctcctgcatga |
| SEQ ID NO: 13 | atgctgctgctggtgacaagcctgctgctgtgcgaactgccccatcccgccttcctgctgattcctg |
| | gtgtacactccgaggtgcagctgttggagtctgggggaggcttggtacagcctggggggtccctg |
| | agactctcctgtgcagcctctggattcacctttagcagctatgccatgagctgggtccgccaggctc |
| | cagggaaggggctggagtgggtctcagctattagtggtagtggtggtagcacatactacgcagac |
| | tccgtgaagggccggttcaccatctccagagacaattccaagaacacgctgtatctgcaaatgaac |
| | agcctgagagccgaggacacggccgtgtattactgtgcgagaggaaagcgatactttgactactg |
| | gggccaggggacaatggtcaccgtctcgagtggtggggggggcagcggtggtggaggctctg |
| | gtggaggagggagctcctatgagctgactcagccaccctcagcgtctgggacccccgggcaga |
| | gggtcaccatctcttgttctggaggcagctccaacatcggaagtaatactgtaaactggttccggca |
| | gctcccaggaacggcccccaaactcctcgtttattttaataatcagcgaccctcaggggtccctga |
| | ccgattctctggctccaagtctggcacctcggcctccctggccatcggtgggctccagtctgacga |
| | tgaggctgactattactgtgtagcatgggatgactctctgaatgctccggtgttcggcggagggac |
| | caaggtcaccgtcctagagagcaaatatggaccaccatgccctccatgtcctttttgggtcctggtg |
| | gtcgtgggaggcgtgctggcatgttattccctgctggtcactgtggccttcatcatcttctgggtgcg |
| | gagcaagcggagccggctgctgcactctgactacatgaacatgactccacggagacccggccct |
| | acccggaaacattatcagccctacgccccacccagagattttgccgcttataggtccagggtgaa |
| | gttttctcgcagtgcagatgcccctgcttatcagcagggacagaatcagctgtacaacgagctgaa |
| | tctgggcaggcgcgaggaatacgacgtgctggataagcgacggggcagagaccccgaaatgg |
| | gagggaagcccagaaggaaaaaccctcaggaggggctgtataatgaactgcagaaggacaaa |
| | atggcagaggcctacagtgaaatcgggatgaagggagagcgccgacggggaaaaggccacg |
| | atggactgtatcagggcctgtctactgccaccaaggacacctacgatgccctgcacatgcaggct |
| | ctgcctccacgctga |
| SEQ ID NO: 14 | atgctgctgctggtgacaagcctgctgctgtgcgaactgccccatcccgccttcctgctgattcctg |
| | gtgtacactccgaggtgcagctgttggagtctgggggaggcttggtacagcctggggggtccctg |
| | agactctcctgtgcagcctctggattcacctttagcagctatgccatgagctgggtccgccaggctc |
| | cagggaaggggctggagtgggtctcagctattagtggtagtggtggtagcacatactacgcagac |
| | tccgtgaagggccggttcaccatctccagagacaattccaagaacacgctgtatctgcaaatgaac |
| | agcctgagagccgaggacacggccgtgtattactgtgcgagaggaaagcgatactttgactactg |
| | gggccaggggacaatggtcaccgtctcgagtggtggggggggcagcggtggtggaggctctg |
| | gtggaggagggagctcctatgagctgactcagccaccctcagcgtctgggacccccgggcaga |
| | gggtcaccatctcttgttctggaggcagctccaacatcggaagtaatactgtaaactggttccggca |
| | gctcccaggaacggcccccaaactcctcgtttattttaataatcagcgaccctcaggggtccctga |
| | ccgattctctggctccaagtctggcacctcggcctccctggccatcggtgggctccagtctgacga |
| | tgaggctgactattactgtgtagcatgggatgactctctgaatgctccggtgttcggcggagggac |
| | caaggtcaccgtcctagagagcaaatatggaccaccatgccctccatgtcctttttgggtcctggtg |
| | gtcgtgggaggcgtgctggcatgttattccctgctggtcactgtggccttcatcatcttctgggtgcg |
| | gagcaagcggagccggctgctgcactctgactacatgaacatgactccacggagacccggccct |
| | acccggaaacattatcagccctacgccccacccagagattttgccgcttataggtccaagcgcgg |
| | ccgaaagaaactgctgtacatcttcaaacagcccttcatgagacccgtccagacaactcaggagg |
| | aagacggctgcagctgtaggttccccgaggaagaggaagggggatgtgagctgagggtgaagt |
| | tttctcgcagtgcagatgcccctgcttatcagcagggacagaatcagctgtacaacgagctgaatc |
| | tgggcaggcgcgaggaatacgacgtgctggataagcgacggggcagagaccccgaaatggg |
| | agggaagcccagaaggaaaaaccctcaggaggggctgtataatgaactgcagaaggacaaaat |
| | ggcagaggcctacagtgaaatcgggatgaagggagagcgccgacggggaaaaggccacgat |
| | ggactgtatcagggcctgtctactgccaccaaggacacctacgatgccctgcacatgcaggctct |
| | gcctccacgctga |
| SEQ ID NO: 15 | atgctgctgctggtgacaagcctgctgctgtgcgaactgccccatcccgccttcctgctgattcctg |
| | aggtccagctgctggagagcggaggaggactggtgcagcctggaggaagtctgcgactgtcat |
| | gcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctggagagcaaatatggaccaccatgccctccatgtcctttttgggtcctggtggtcgtg |
| | ggaggcgtgctggcatgttattctctgctggtcacagtggctttcatcatcttctgggtcaagcgagg |
| | ccggaagaaactgctgtacatcttcaaacagccttttatgcgcccagtgcagacaactcaggagg |
| | aagacggctgctcttgtcggttccccgaggaagaggaagggggatgtgagctgcgcgtgaagtt |
| | ttctcgaagtgccgatgctcctgcatatcagcagggacagaaccagctgtacaacgagctgaatct |
| | gggccggagagaggaatacgacgtgctggataagaggcgcggcagagacccagaaatgggc |
| | gggaagccacgacggaaaaacccccaggaggggctgtataatgaactgcagaaggacaaaat |
| | ggccgaggcttacagcgaaatcgggatgaagggagagagaaggcgcggaaaaggccacgat |
| | ggactgtatcagggcctgagcactgccaccaaggacacctacgatgctctgcacatgcaggcac |
| | tgccacccaggtga |
| SEQ ID NO: 16 | atgctgctgctggtgacaagcctgctgctgtgcgaactgccccatcccgccttcctgctgattcctg |
| | aggtccagctgctggagagcggaggaggactggtgcagcctggaggaagtctgcgactgtcat |
| | gcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctggagagcaaatatggaccaccatgccctccatgtcctttttgggtcctggtggtcgtg |
| | ggaggcgtgctggcatgttactccctgctggtcactgtggccttcatcatcttctgggtgcgggtga |
| | agttttctcgcagtgccgacgctcccgcatga |
| SEQ ID NO: 17 | atgctgctgctggtgacaagcctgctgctgtgcgaactgccccatcccgccttcctgctgattcctg |
| | aggtccagctgctggagagcggaggaggactggtgcagcctggaggaagtctgcgactgtcat |
| | gcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctggagagcaaatatggaccaccatgccctccatgtcctttttgggtcctggtggtcgtg |
| | ggaggcgtgctggcatgttattccctgctggtcacagtggccttcatcatcttctgggtgcggagca |
| | agcggagccggctgctgcactctgactacatgaacatgaccccccggagacccggccctacaa |
| | gaaagcattatcagccttacgccccacccagggacttcgcagcttatcgctcccgagtgaaattttc |
| | tcgcagtgcagatgcccccgcttatcagcagggccagaatcagctgtacaacgagctgaatctgg |
| | ggaggcgcgaggaatacgacgtgctggataagcgacggggccgggaccccgaaatgggagg |
| | aaagcctagaaggaaaaacccacaggagggcctgtataatgaactgcagaaggacaaaatggc |
| | agaggcctacagcgaaatcggaatgaagggagagcgccgacggggcaaaggacacgatggc |
| | ctgtatcaggggctgagcaccgccacaaaggacacctacgatgccctgcacatgcaggctctgc |
| | ctccacgctga |
| SEQ ID NO: 18 | atgctgctgctggtgacaagcctgctgctgtgcgaactgccccatcccgccttcctgctgattcctg |
| | aggtccagctgctggagagcggaggaggactggtgcagcctggaggaagtctgcgactgtcat |
| | gcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctggagagcaaatatggaccaccatgccctccatgtcctttttgggtcctggtggtcgtg |
| | ggaggcgtgctggcatgttattccctgctggtcactgtggccttcatcatcttctgggtgcggagca |
| | agcggagccggctgctgcactctgactacatgaacatgactccacggagacccggccctacccg |
| | gaaacattatcagccctacgccccacccagagattttgccgcttataggtccaagcgcggccgaa |
| | agaaactgctgtacatcttcaaacagcccttcatgagacccgtccagacaactcaggaggaagac |
| | ggctgcagctgtaggttccccgaggaagaggaagggggatgtgagctgagggtgaagttttctc |
| | gcagtgcagatgcccctgcttatcagcagggacagaatcagctgtacaacgagctgaatctgggc |
| | aggcgcgaggaatacgacgtgctggataagcgacggggcagagaccccgaaatgggaggga |
| | agcccagaaggaaaaaccctcaggaggggctgtataatgaactgcagaaggacaaaatggcag |
| | aggcctacagtgaaatcgggatgaagggagagcgccgacggggaaaaggccacgatggactg |
| | tatcagggcctgtctactgccaccaaggacacctacgatgccctgcacatgcaggctctgcctcca |
| | cgctga |
| SEQ ID NO: 19 | MLLLVTSLLLCELPHPAFLLIPDVVMTQSPLSLPVTPGEPASIS |
| | CRSSQSLVHSNRNTYLHWYLQKPGQSPQLLIYKVSNRFSGVP |
| | DRFSGSGSGTDFTLKISRVEAEDVGVYYCSQNTHVPPTFGQGT |
| | KLEIKGGGGSGGGGSGGGGSQVQLVQSGAEVKKPGASVKVS |
| | CKASGYTFTDYEMHWVRQAPGQGLEWMGALDPKTGDTAYS |
| | QKFKGRVTLTADKSTSTAYMELSSLTSEDTAVYYCTRFYSYT |
| | YWGQGTLVTVSSDKTHTCPPCPFWVLVVVGGVLACYSLLVT |
| | VAFIIFWVKRGRKKLLYIFKQPFMRPVQTTQEEDGCSCRFPEE |
| | EEGGCELRVKFSRSADAPAYQQGQNQLYNELNLGRREEYDV |
| | LDKRRGRDPEMGGKPQRRKNPQEGLYNELQKDKMAEAYSEI |
| | GMKGERRRGKGHDGLYQGLSTATKDTYDALHMQALPPR* |
| SEQ ID NO: 20 | MLLLVTSLLLCELPHPAFLLIPDVVMTQSPLSLPVTPGEPASIS |
| | CRSSQSLVHSNRNTYLHWYLQKPGQSPQLLIYKVSNRFSGVP |
| | DRFSGSGSGTDFTLKISRVEAEDVGVYYCSQNTHVPPTFGQGT |
| | KLEIKGGGGSGGGGSGGGGSQVQLVQSGAEVKKPGASVKVS |
| | CKASGYTFTDYEMHWVRQAPGQGLEWMGALDPKTGDTAYS |
| | QKFKGRVTLTADKSTSTAYMELSSLTSEDTAVYYCTRFYSYT |
| | YWGQGTLVTVSSDKTHTCPPCPFWVLVVVGGVLACYSLLVT |
| | VAFIIFWVRSKRSRLLHSDYMNMTPRRPGPTRKHYQPYAPPR |
| | DFAAYRSKRGRKKLLYIFKQPFMRPVQTTQEEDGCSCRFPEEE |
| | EGGCELRVKFSRSADAPAYQQGQNQLYNELNLGRREEYDVL |
| | DKRRGRDPEMGGKPQRRKNPQEGLYNELQKDKMAEAYSEIG |
| | MKGERRRGKGHDGLYQGLSTATKDTYDALHMQALPPR* |
| SEQ ID NO: 21 | MLLLVTSLLLCELPHPAFLLIPQVQLVQSGGGVVQPGRSLRLS |
| | CAASGFTFSSYGLHWVRQAPGKGLEWVAAISYDGSKKYYAD |
| | SVKGRLTISRDNSKNTLYLQMNSLRPDDTALYFCARGWFVEP |
| | LSWGQGTLVTVSSGGGGSGGGGSGGGGSQSVLTQPPSASGTP |
| | GQRVTISCSGSSSNIGSNTVNWYQQLPGTAPKLLIYSNNQRPS |
| | GVPDRFSGSKSGTSASLAISGLQSEDEADYYCAAWDDSLNGY |
| | VFGTGTKLTVLESKYGPPCPPCPFWVLVVVGGVLACYSLLVT |
| | VAFIIFWVKRGRKKLLYIFKQPFMRPVQTTQEEDGCSCRFPEE |
| | EEGGCELRVKFSRSADAPAYQQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR* |
| SEQ ID NO: 22 | atgctgctgctggtgacaagcctgctgctgtgcgaactgccccatcccgccttcctgctgattcctg |
| | atgtcgtgatgacgcagagccctctctctcttcccgttacccctggtgaacccgcatcaataagttg |
| | ccgctccagtcaatcacttgtacattcaaatcgcaatacctacctgcactggtatttgcagaagccg |
| | ggacaatcccctcaattgttgatatataaggtatccaatcgcttttctggagttcctgatagattcagc |
| | ggatccgggtctggtactgatttcactctgaaaatatccagggtcgaagctgaggacgtaggcgta |
| | tattattgctctcagaacacgcatgtcccgccgactttcggccagggcactaaacttgagatcaagg |
| | gtggggggggcagcggtggtggaggctctggtggaggagggagccaggtccaactcgttcaa |
| | agtggcgcagaggtcaaaaagccaggcgcgagcgttaaagtatcatgtaaggccagcggttata |
| | ctttcactgattatgaaatgcactgggtgcgacaagcccccgggcaaggtcttgagtggatgggtg |
| | cacttgatccaaaaactggggatactgcctatagccagaaattcaaagggcgcgtcacactcactg |
| | ccgacaaaagtacgagcacagcttatatggaattgagttcactgacgagcgaggatacggcagtt |
| | tattactgtacgcgcttctactcttacacttattgggggcaaggcactttggttactgtgtcctctgaca |
| | agacccatacgtgtccaccgtgtcccttctgggtattggttgtggtcggcggtgtccttgcttgttaca |
| | gccttctcgtgacagtcgcattcataattttttgggtgaaaagaggtcggaaaaagttgctgtatatttt |
| | caaacaaccctttatgagacctgtacaaacgactcaggaagaggatggttgtagttgcaggtttcc |
| | ggaggaggaggaaggtgggtgcgaactgcgggtgaaatttagtagaagcgctgacgcaccag |
| | cttaccaacaaggacagaaccaattgtacaacgagcttaacttgggtaggagggaggaatatgat |
| | gtactggacaaaaggcgaggtcgcgatccggaaatgggaggcaagccacagcgccggaaaaa |
| | cccgcaggaaggcttgtacaacgaacttcagaaagataaaatggcagaagcatactccgaaata |
| | gggatgaaaggtgaacggcggcgaggcaagggccacgacggtctgtaccaagggttgtcaac |
| | ggcaactaaagacacgtatgatgcacttcatatgcaagctctgccacccaggtga |
| SEQ ID NO: 23 | atgctgctgctggtgacaagcctgctgctgtgcgaactgccccatcccgccttcctgctgattcctg |
| | atgtcgtgatgacgcagagccctctctctcttcccgttacccctggtgaacccgcatcaataagttg |
| | ccgctccagtcaatcacttgtacattcaaatcgcaatacctacctgcactggtatttgcagaagccg |
| | ggacaatcccctcaattgttgatatataaggtatccaatcgcttttctggagttcctgatagattcagc |
| | ggatccgggtctggtactgatttcactctgaaaatatccagggtcgaagctgaggacgtaggcgta |
| | tattattgctctcagaacacgcatgtcccgccgactttcggccagggcactaaacttgagatcaagg |
| | gtggggggggcagcggtggtggaggctctggtggaggagggagccaggtccaactcgttcaa |
| | agtggcgcagaggtcaaaaagccaggcgcgagcgttaaagtatcatgtaaggccagcggttata |
| | ctttcactgattatgaaatgcactgggtgcgacaagcccccgggcaaggtcttgagtggatgggtg |
| | cacttgatccaaaaactggggatactgcctatagccagaaattcaaagggcgcgtcacactcactg |
| | ccgacaaaagtacgagcacagcttatatggaattgagttcactgacgagcgaggatacggcagtt |
| | tattactgtacgcgcttctactcttacacttattgggggcaaggcactttggttactgtgtcctctgaca |
| | agacccatacgtgtccaccgtgtcccttctgggtattggttgtggtcggcggtgtccttgcttgttaca |
| | gccttctcgtgacagtcgcattcataattttttgggtgcggagcaagcggagccggctgctgcactc |
| | tgactacatgaacatgactccacggagacccggccctacccggaaacattatcagccctacgccc |
| | cacccagagattttgccgcttataggtccaaaagaggtcggaaaaagttgctgtatattttcaaaca |
| | accctttatgagacctgtacaaacgactcaggaagaggatggttgtagttgcaggtttccggagga |
| | ggaggaaggtgggtgcgaactgcgggtgaaatttagtagaagcgctgacgcaccagcttaccaa |
| | caaggacagaaccaattgtacaacgagcttaacttgggtaggagggaggaatatgatgtactgga |
| | caaaaggcgaggtcgcgatccggaaatgggaggcaagccacagcgccggaaaaacccgcag |
| | gaaggcttgtacaacgaacttcagaaagataaaatggcagaagcatactccgaaatagggatgaa |
| | aggtgaacggcggcgaggcaagggccacgacggtctgtaccaagggttgtcaacggcaactaa |
| | agacacgtatgatgcacttcatatgcaagctctgccacccaggtga |
| SEQ ID NO: 24 | atgctgctgctggtgacaagcctgctgctgtgcgaactgccccatcccgccttcctgctgattcctc |
| | aggtccagcttgtgcaaagcggaggaggagtggtacagcctggccgctctttgagactgtcttgt |
| | gcggccagtggatttacattctcttcttatgggttgcattgggtcagacaagcaccgggcaaaggat |
| | tggaatgggtcgcggccattagctatgatggctcaaagaaatattatgccgattccgtaaaaggga |
| | ggttgacaataagccgggataacagcaagaacactttgtatcttcagatgaatagcctccgaccgg |
| | acgacacggcactgtatttttgcgcacgcgggtggtttgtagaacccctgagttggggacaaggta |
| | ctcttgtcacggtatcttctggcggaggtgggagtggtgggggtggcagtggcgggggtgggtc |
| | acaaagcgtgcttacacaacctccttctgcgagcggaactccgggacaacgggttacgatttcatg |
| | ctccggctcaagtagcaatataggatcaaatacagtgaattggtatcaacaactccctggcacagc |
| | gcccaagctgctgatctactctaataaccagaggccgagtggtgtgccagataggttcagtggctc |
| | taaatcaggtactagcgcgagcctcgccatttcaggacttcaatcagaggatgaagcggactacta |
| | ctgtgccgcgtgggatgattcacttaatggatatgttttcgggaccggaacaaaattgacggtattg |
| | gagagcaaatatggaccaccatgccctccatgtcctttttgggtcctggtggtcgtgggaggcgtg |
| | ctggcatgttattctctgctggtcacagtggctttcatcatcttctgggtcaagcgaggccggaagaa |
| | actgctgtacatcttcaaacagccttttatgcgcccagtgcagacaactcaggaggaagacggctg |
| | ctcttgtcggttccccgaggaagaggaagggggatgtgagctgcgcgtgaagttttctcgaagtg |
| | ccgatgctcctgcatatcagcagggacagaaccagctgtacaacgagctgaatctgggccggag |
| | agaggaatacgacgtgctggataagaggcgcggcagagacccagaaatgggcgggaagcca |
| | cgacggaaaaacccccaggaggggctgtataatgaactgcagaaggacaaaatggccgaggct |
| | tacagcgaaatcgggatgaagggagagagaaggcgcggaaaaggccacgatggactgtatca |
| | gggcctgagcactgccaccaaggacacctacgatgctctgcacatgcaggcactgccacccag |
| | gTGA |
| SEQ ID NO: 25 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCARGKRYFD |
| | YWGQGTMVTVSSGGGGSGGGGSGGGGSSYELTQPPSASGTP |
| | GQRVTISCSGGSSNIGSNTVNWFRQLPGTAPKLLVYFNNQRPS |
| | GVPDRFSGSKSGTSASLAIGGLQSDDEADYYCVAWDDSLNAP |
| | VFGGGTKVTVLESKYGPPCPPCPFWVLVVVGGVLACYSLLVT |
| | VAFIIFWVKRGRKKLLYIFKQPFMRPVQTTQEEDGCSCRFPEE |
| | EEGGCELRVKFSRSADAPAYQQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR |
| SEQ ID NO: 26 | ATGCTGCTGCTGGTGACAAGCCTGCTGCTGTGCGAACTGCC |
| | CCATCCCGCCTTCCTGCTGATTCCTGAGGTGCAGCTGTTGG |
| | AGTCTGGGGGAGGCTTGGTACAGCCTGGGGGGTCCCTGAG |
| | ACTCTCCTGTGCAGCCTCTGGATTCACCTTTAGCAGCTATG |
| | CCATGAGCTGGGTCCGCCAGGCTCCAGGGAAGGGGCTGGA |
| | GTGGGTCTCAGCTATTAGTGGTAGTGGTGGTAGCACATACT |
| | ACGCAGACTCCGTGAAGGGCCGGTTCACCATCTCCAGAGA |
| | CAATTCCAAGAACACGCTGTATCTGCAAATGAACAGCCTG |
| | AGAGCCGAGGACACGGCCGTGTATTACTGTGCGAGAGGAA |
| | AGCGATACTTTGACTACTGGGGCCAGGGGACAATGGTCAC |
| | CGTCTCGAGTGGTGGGGGGGGCAGCGGTGGTGGAGGCTCT |
| | GGTGGAGGAGGGAGCTCCTATGAGCTGACTCAGCCACCCT |
| | CAGCGTCTGGGACCCCCGGGCAGAGGGTCACCATCTCTTGT |
| | TCTGGAGGCAGCTCCAACATCGGAAGTAATACTGTAAACT |
| | GGTTCCGGCAGCTCCCAGGAACGGCCCCCAAACTCCTCGTT |
| | TATTTTAATAATCAGCGACCCTCAGGGGTCCCTGACCGATT |
| | CTCTGGCTCCAAGTCTGGCACCTCGGCCTCCCTGGCCATCG |
| | GTGGGCTCCAGTCTGACGATGAGGCTGACTATTACTGTGTA |
| | GCATGGGATGACTCTCTGAATGCTCCGGTGTTCGGCGGAG |
| | GGACCAAGGTCACCGTCCTAGAGAGCAAATATGGACCACC |
| | ATGCCCTCCATGTCCTTTTTGGGTCCTGGTGGTCGTGGGAG |
| | GCGTGCTGGCATGTTATTCTCTGCTGGTCACAGTGGCTTTC |
| | ATCATCTTCTGGGTCAAGCGAGGCCGGAAGAAACTGCTGT |
| | ACATCTTCAAACAGCCTTTTATGCGCCCAGTGCAGACAACT |
| | CAGGAGGAAGACGGCTGCTCTTGTCGGTTCCCCGAGGAAG |
| | AGGAAGGGGGATGTGAGCTGCGCGTGAAGTTTTCTCGAAG |
| | TGCCGATGCTCCTGCATATCAGCAGGGACAGAACCAGCTG |
| | TACAACGAGCTGAATCTGGGCCGGAGAGAGGAATACGACG |
| | TGCTGGATAAGAGGCGCGGCAGAGACCCAGAAATGGGCG |
| | GGAAGCCACGACGGAAAAACCCCCAGGAGGGGCTGTATA |
| | ATGAACTGCAGAAGGACAAAATGGCCGAGGCTTACAGCGA |
| | AATCGGGATGAAGGGAGAGAGAAGGCGCGGAAAAGGCCA |
| | CGATGGACTGTATCAGGGCCTGAGCACTGCCACCAAGGAC |
| | ACCTACGATGCTCTGCACATGCAGGCACTGCCACCCAGG |
| SEQ ID NO: 27 | EVQLLESGGGLVQPGGSLRLSCAASGFTFSSYAMSWVRQAPG |
| | KGLEWVSAISGSGGSTYYADSVKGRFTISRDNSKNTLYLQMN |
| | SLRAEDTAVYYCARGKRYFDYWGQGTMVTVSS |
| SEQ ID NO: 28 | SYELTQPPSASGTPGQRVTISCSGGSSNIGSNTVNWFRQLPGT |
| | APKLLVYFNNQRPSGVPDRFSGSKSGTSASLAIGGLQSDDEAD |
| | YYCVAWDDSLNAPVFGGGTKVTVL |
| SEQ ID NO: 29 | EVQLLESGGGLVQPGGSLRLSCAASGFTFSSYAMSWVRQAPG |
| | KGLEWVSAISGSGGSTYYADSVKGRFTISRDNSKNTLYLQMN |
| | SLRAEDTAVYYCAKGKRYFDYWGQGTMVTVSS |
| SEQ ID NO: 30 | QSVLTQPPSASGTPGQRVTISCSGGSSDIGSNTVNWYQQLPGT |
| | APKLLIYYNNQRPSGVPDRFSGSKSGTSASLAISGLQSEDEAD |
| | YYCATWDDRMYSPVFGGGTKLTVL |
| SEQ ID NO: 31 | DVVMTQSPLSLPVTPGEPASISCRSSQSLVHSNRNTYLHWYLQ |
| | KPGQSPQLLIYKVSNRFSGVPDRFSGSGSGTDFTLKISRVEAED |
| | VGVYYCSQNTHVPPTFGQGTKLEIKGGGGSGGGGSGGGGSQ |
| | VQLVQSGAEVKKPGASVKVSCKASGYTFTDYEMHWVRQAP |
| | GQGLEWMGALDPKTGDTAYSQKFKGRVTLTADKSTSTAYM |
| | ELSSLTSEDTAVYYCTRFYSYTYWGQGTLVTVSS |
| SEQ ID NO: 32 | QVQLVQSGGGVVQPGRSLRLSCAASGFTFSSYGLHWVRQAP |
| | GKGLEWVAAISYDGSKKYYADSVKGRLTISRDNSKNTLYLQ |
| | MNSLRPDDTALYFCARGWFVEPLSWGQGTLVTVSSGGGGSG |
| | GGGSGGGGSQSVLTQPPSASGTPGQRVTISCSGSSSNIGSNTV |
| | NWYQQLPGTAPKLLIYSNNQRPSGVPDRFSGSKSGTSASLAIS |
| | GLQSEDEADYYCAAWDDSLNGYVFGTGTKLTVL |
| SEQ ID NO: 33 | gaggtgcagctgttggagtctgggggaggcttggtacagcctggggggtccctgagactctcctg |
| | tgcagcctctggattcacctttagcagctatgccatgagctgggtccgccaggctccagggaagg |
| | ggctggagtgggtctcagctattagtggtagtggtggtagcacatactacgcagactccgtgaag |
| | ggccggttcaccatctccagagacaattccaagaacacgctgtatctgcaaatgaacagcctgag |
| | agccgaggacacggccgtgtattactgtgcgagaggaaagcgatactttgactactggggccag |
| | gggacaatggtcaccgtctcgagtggtggggggggcagcggtggtggaggctctggtggagg |
| | agggagctcctatgagctgactcagccaccctcagcgtctgggacccccgggcagagggtcac |
| | catctcttgttctggaggcagctccaacatcggaagtaatactgtaaactggttccggcagctccca |
| | ggaacggcccccaaactcctcgtttattttaataatcagcgaccctcaggggtccctgaccgattct |
| | ctggctccaagtctggcacctcggcctccctggccatcggtgggctccagtctgacgatgaggct |
| | gactattactgtgtagcatgggatgactctctgaatgctccggtgttcggcggagggaccaaggtc |
| | accgtccta |
| SEQ ID NO: 34 | gaggtccagctgctggagagcggaggaggactggtgcagcctggaggaagtctgcgactgtca |
| | tgcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctg |
| SEQ ID NO: 35 | gatgtcgtgatgacgcagagccctctctctcttcccgttacccctggtgaacccgcatcaataagtt |
| | gccgctccagtcaatcacttgtacattcaaatcgcaatacctacctgcactggtatttgcagaagcc |
| | gggacaatcccctcaattgttgatatataaggtatccaatcgcttttctggagttcctgatagattcag |
| | cggatccgggtctggtactgatttcactctgaaaatatccagggtcgaagctgaggacgtaggcgt |
| | atattattgctctcagaacacgcatgtcccgccgactttcggccagggcactaaacttgagatcaag |
| | ggtggggggggcagcggtggtggaggctctggtggaggagggagccaggtccaactcgttca |
| | aagtggcgcagaggtcaaaaagccaggcgcgagcgttaaagtatcatgtaaggccagcggttat |
| | actttcactgattatgaaatgcactgggtgcgacaagcccccgggcaaggtcttgagtggatgggt |
| | gcacttgatccaaaaactggggatactgcctatagccagaaattcaaagggcgcgtcacactcact |
| | gccgacaaaagtacgagcacagcttatatggaattgagttcactgacgagcgaggatacggcagt |
| | ttattactgtacgcgcttctactcttacacttattgggggcaaggcactttggttactgtgtcctct |
| SEQ ID NO: 36 | caggtccagcttgtgcaaagcggaggaggagtggtacagcctggccgctctttgagactgtcttgt |
| | gcggccagtggatttacattctcttcttatgggttgcattgggtcagacaagcaccgggcaaaggat |
| | tggaatgggtcgcggccattagctatgatggctcaaagaaatattatgccgattccgtaaaaggga |
| | ggttgacaataagccgggataacagcaagaacactttgtatcttcagatgaatagcctccgaccgg |
| | acgacacggcactgtatttttgcgcacgcgggtggtttgtagaacccctgagttggggacaaggta |
| | ctcttgtcacggtatcttctggcggaggtgggagtggtgggggtggcagtggcgggggtgggtc |
| | acaaagcgtgcttacacaacctccttctgcgagcggaactccgggacaacgggttacgatttcatg |
| | ctccggctcaagtagcaatataggatcaaatacagtgaattggtatcaacaactccctggcacagc |
| | gcccaagctgctgatctactctaataaccagaggccgagtggtgtgccagataggttcagtggctc |
| | taaatcaggtactagcgcgagcctcgccatttcaggacttcaatcagaggatgaagcggactacta |
| | ctgtgccgcgtgggatgattcacttaatggatatgttttcgggaccggaacaaaattgacggtattg |
| SEQ ID NO: 37 | GFTFSSYAMS |
| SEQ ID NO: 38 | AISGSGGSTYYADSVKG |
| SEQ ID NO: 39 | GKRYFDY |
| SEQ ID NO: 40 | SGGSSNIGSNTVN |
| SEQ ID NO: 41 | FNNQRPS |
| SEQ ID NO: 42 | VAWDDSLNAPV |
| SEQ ID NO: 43 | SGGSSDIGSNTVN |
| SEQ ID NO: 44 | YNNQRPS |
| SEQ ID NO: 45 | ATWDDRMYSPV |

1. An isolated nucleic acid sequence encoding a chimeric antigen receptor (CAR), wherein the CAR comprises an antigen binding domain specific for glypican 3 (GPC3), wherein the antigen binding domain has an equilibrium dissociation constant (K_{D}) of about 100 nanomolar (nM) or less, wherein the CAR construct does not induce cytokine production in GPC3-cells, and wherein the encoded CAR antigen binding domain comprises the nucleic acid sequence of SEQ ID NO: 33 or SEQ ID NO: 34.

2. The isolated nucleic acid sequence of claim 1, wherein the encoded CAR antigen binding domain comprises an antibody or antigen-binding fragment thereof

3. The isolated nucleic acid sequence of claim 2, wherein the encoded CAR antigen binding domain is a Fab or a single chain variable fragment (scFv).

4. The isolated nucleic acid sequence of claim 3, wherein the antigen binding domain is an scFv.

5. The isolated nucleic acid sequence of claim 1 further encoding a transmembrane domain, a costimulatory domain, and a signal domain.

6. The isolated nucleic acid sequence of claim 5, wherein the encoded transmembrane domain comprises a CD28 transmembrane domain.

7. The isolated nucleic acid sequence of claim 5, wherein the encoded costimulatory domain comprises one or more of CD28, 4-1BB, CD3zeta, OX-40, ICOS, CD27, GITR, and MyD88/CD40 costimulatory domains.

8. The isolated nucleic acid sequence of claim 5, wherein the encoded costimulatory domain comprises one or more of CD28, 4-1BB, and CD3zeta costimulatory domains.

9. The isolated nucleic acid sequence of claim 5, wherein the encoded signal domain comprises a sequence encoding a CSFR2 signal peptide.

10. The isolated nucleic acid sequence of claim 1 further encoding a hinge/spacer domain.

11. The isolated nucleic acid sequence of claim 10, wherein the encoded hinge/spacer domain is an IgG4P hinge/spacer.

12. The isolated nucleic acid sequence of claim 1, wherein the nucleic acid sequence comprises SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 26.

13. An anti-GPC3 chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39; and
wherein the VL comprises a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

14. The anti-GPC3 CAR of claim 13, wherein the VH comprises the amino acid sequence of SEQ ID NO: 27 or SEQ ID NO: 29.

15. The anti-GPC3 CAR of claim 13, wherein the VL comprises the amino acid sequence of SEQ ID NO: 28 or SEQ ID NO: 30.

16. The anti-GPC3 CAR of claim 13, wherein the CAR further comprises a transmembrane domain, a costimulatory domain, and a signal domain.

17. The anti-GPC3 CAR of claim 16, wherein the CAR comprises the amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25.

18. A vector comprising a nucleic acid sequence encoding a chimeric antigen receptor (CAR), wherein the nucleic acid sequence comprises SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

19. A cell comprising the vector of claim 18.

20. A cell comprising a nucleic acid sequence encoding a chimeric antigen receptor (CAR), wherein the CAR comprises an antigen binding domain specific for glypican 3 (GPC3), wherein the antigen binding domain has an equilibrium dissociation constant (K_{D}) of about 100 nanomolar (nM) or less, and wherein the CAR construct does not induce cytokine production in GPC3-cells.

21. The cell of claim 20, wherein the nucleic acid sequence comprises SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

22. A cell comprising an anti-GPC3 chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39, and
wherein the VL comprises a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

23. The cell of claim 22, wherein the VH comprises the amino acid sequence of SEQ ID NO: 27 or SEQ ID NO: 29.

24. The cell of claim 22, wherein the VL comprises the amino acid sequence of SEQ ID NO: 28 or SEQ ID NO: 30.

25. The cell of claim 22, wherein the CAR further comprises a transmembrane domain, a costimulatory domain, and a signal domain.

26. The cell of claim 22, wherein the CAR comprises the amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25.

27. The cell of claim 22, wherein the cell is selected from the group consisting of a T cell, a Natural Killer (NK) cell, a cytotoxic T lymphocyte (CTL), and a regulatory T cell.

28. The cell of claim 27, wherein the cell exhibits an anti-tumor immunity upon contacting a tumor cell expressing GPC3.

29. A method of treating cancer, comprising:
administering to a subject in need thereof an effective amount of a cell comprising an anti-GPC3 chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39, and
wherein the VL comprises a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

30. The method of claim 29 further comprising inhibiting tumor growth, inducing tumor regression, and/or prolonging survival of the subject.

31. The method of claim 29, wherein the cell is an autologous cell.

32. The method of claim 31, wherein the autologous cell is selected from the group consisting of a T cell, a Natural Killer (NK) cell, a cytotoxic T lymphocyte (CTL), and a regulatory T cell.

33. The method of claim 29, wherein the cancer is a solid tumor.

34. The method of claim 33, wherein the cancer is hepatocellular carcinoma, non-small cell lung cancer, ovarian cancer, and/or squamous cell lung carcinoma.

35. The method of claim 34, wherein the cancer is hepatocellular carcinoma.

36. The method of claim 29 further comprising administrating to the subject an effective amount of an anti-TNFα antibody.


SEQUENCE LISTING (46 sequences)

SEQ ID NO: 1 - protein, 241 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVL

SEQ ID NO: 2 - protein, 241 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVL

SEQ ID NO: 3 - protein, 460 aa, Artificial Sequence
  Description of Artificial Sequence Synthetic polypeptide
MLLLVTSLLLCELPHPAFLLIPGVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 318 aa, Artificial Sequence
  Description of Artificial Sequence Synthetic polypeptide
MLLLVTSLLLCELPHPAFLLIPGVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVRVKFSRSADAPA

SEQ ID NO: 5 - protein, 459 aa, Artificial Sequence
  Description of Artificial Sequence Synthetic polypeptide
MLLLVTSLLLCELPHPAFLLIPGVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 501 aa, Artificial Sequence
  Description of Artificial Sequence Synthetic polypeptide
MLLLVTSLLLCELPHPAFLLIPGVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 456 aa, Artificial Sequence
  Description of Artificial Sequence Synthetic polypeptide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 314 aa, Artificial Sequence
  Description of Artificial Sequence Synthetic polypeptide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLESKYGPPCPPCPFWVLVVVGGVLACYSLLVTVAFIIFWVRVKFSRSADAPA

SEQ ID NO: 9 - protein, 455 aa, Artificial Sequence
  Description of Artificial Sequence Synthetic polypeptide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 497 aa, Artificial Sequence
  Description of Artificial Sequence Synthetic polypeptide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLESKYGPPCPPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1383 bp, Artificial Sequence
  Description of Artificial Sequence Synthetic polynucleotide
atgctgctgctggtgacaagcctgctgctgtgcgaactgccccatcccgccttcctgctgattcctggtgtacactccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattctctgctggtcacagtggctttcatcatcttctgggtcaagcgaggccggaagaaactgctgtacatcttcaaacagccttttatgcgcccagtgcagacaactcaggaggaagacggctgctcttgtcggttccccgaggaagaggaagggggatgtgagctgcgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccaggtga

SEQ ID NO: 12 - DNA, 957 bp, Artificial Sequence
  Description of Artificial Sequence Synthetic polynucleotide
atgctgctgctggtgacaagcctgctgctgtgcgaactgccccatcccgccttcctgctgattcctggtgtacactccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattctctgctggtcacagtggctttcatcatcttctgggtccgcgtgaagttttctcgaagtgccgatgctcctgcatga

SEQ ID NO: 13 - DNA, 1380 bp, Artificial Sequence
  Description of Artificial Sequence Synthetic polynucleotide
atgctgctgctggtgacaagcctgctgctgtgcgaactgccccatcccgccttcctgctgattcctggtgtacactccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattccctgctggtcactgtggccttcatcatcttctgggtgcggagcaagcggagccggctgctgcactctgactacatgaacatgactccacggagacccggccctacccggaaacattatcagccctacgccccacccagagattttgccgcttataggtccagggtgaagttttctcgcagtgcagatgcccctgcttatcagcagggacagaatcagctgtacaacgagctgaatctgggcaggcgcgaggaatacgacgtgctggataagcgacggggcagagaccccgaaatgggagggaagcccagaaggaaaaaccctcaggaggggctgtataatgaactgcagaaggacaaaatggcagaggcctacagtgaaatcgggatgaagggagagcgccgacggggaaaaggccacgatggactgtatcagggcctgtctactgccaccaaggacacctacgatgccctgcacatgcaggctctgcctccacgctga

SEQ ID NO: 14 - DNA, 1506 bp, Artificial Sequence
  Description of Artificial Sequence Synthetic polynucleotide
atgctgctgctggtgacaagcctgctgctgtgcgaactgccccatcccgccttcctgctgattcctggtgtacactccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattccctgctggtcactgtggccttcatcatcttctgggtgcggagcaagcggagccggctgctgcactctgactacatgaacatgactccacggagacccggccctacccggaaacattatcagccctacgccccacccagagattttgccgcttataggtccaagcgcggccgaaagaaactgctgtacatcttcaaacagcccttcatgagacccgtccagacaactcaggaggaagacggctgcagctgtaggttccccgaggaagaggaagggggatgtgagctgagggtgaagttttctcgcagtgcagatgcccctgcttatcagcagggacagaatcagctgtacaacgagctgaatctgggcaggcgcgaggaatacgacgtgctggataagcgacggggcagagaccccgaaatgggagggaagcccagaaggaaaaaccctcaggaggggctgtataatgaactgcagaaggacaaaatggcagaggcctacagtgaaatcgggatgaagggagagcgccgacggggaaaaggccacgatggactgtatcagggcctgtctactgccaccaaggacacctacgatgccctgcacatgcaggctctgcctccacgctga

SEQ ID NO: 15 - DNA, 1371 bp, Artificial Sequence
  Description of Artificial Sequence Synthetic polynucleotide
atgctgctgctggtgacaagcctgctgctgtgcgaactgccccatcccgccttcctgctgattcctgaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctggagagcaaatatggaccaccatgccctccatgtcctttttgggtcctggtggtcgtgggaggcgtgctggcatgttattctctgctggtcacagtggctttcatcatcttctgggtcaagcgaggccggaagaaactgctgtacatcttcaaacagccttttatgcgcccagtgcagacaactcaggaggaagacggctgctcttgtcggttccccgaggaagaggaagggggatgtgagctgcgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccaggtga

SEQ ID NO: 16 - DNA, 945 bp, Artificial Sequence
  Description of Artificial Sequence Synthetic polynucleotide
atgctgctgctggtgacaagcctgctgctgtgcgaactgccccatcccgccttcctgctgattcctgaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctggagagcaaatatggaccaccatgccctccatgtcctttttgggtcctggtggtcgtgggaggcgtgctggcatgttactccctgctggtcactgtggccttcatcatcttctgggtgcgggtgaagttttctcgcagtgccgacgctcccgcatga

SEQ ID NO: 17 - DNA, 1368 bp, Artificial Sequence
  Description of Artificial Sequence Synthetic polynucleotide
atgctgctgctggtgacaagcctgctgctgtgcgaactgccccatcccgccttcctgctgattcctgaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctggagagcaaatatggaccaccatgccctccatgtcctttttgggtcctggtggtcgtgggaggcgtgctggcatgttattccctgctggtcacagtggccttcatcatcttctgggtgcggagcaagcggagccggctgctgcactctgactacatgaacatgaccccccggagacccggccctacaagaaagcattatcagccttacgccccacccagggacttcgcagcttatcgctcccgagtgaaattttctcgcagtgcagatgcccccgcttatcagcagggccagaatcagctgtacaacgagctgaatctggggaggcgcgaggaatacgacgtgctggataagcgacggggccgggaccccgaaatgggaggaaagcctagaaggaaaaacccacaggagggcctgtataatgaactgcagaaggacaaaatggcagaggcctacagcgaaatcggaatgaagggagagcgccgacggggcaaaggacacgatggcctgtatcaggggctgagcaccgccacaaaggacacctacgatgccctgcacatgcaggctctgcctccacgctga

SEQ ID NO: 18 - DNA, 1494 bp, Artificial Sequence
  Description of Artificial Sequence Synthetic polynucleotide
atgctgctgctggtgacaagcctgctgctgtgcgaactgccccatcccgccttcctgctgattcctgaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctggagagcaaatatggaccaccatgccctccatgtcctttttgggtcctggtggtcgtgggaggcgtgctggcatgttattccctgctggtcactgtggccttcatcatcttctgggtgcggagcaagcggagccggctgctgcactctgactacatgaacatgactccacggagacccggccctacccggaaacattatcagccctacgccccacccagagattttgccgcttataggtccaagcgcggccgaaagaaactgctgtacatcttcaaacagcccttcatgagacccgtccagacaactcaggaggaagacggctgcagctgtaggttccccgaggaagaggaagggggatgtgagctgagggtgaagttttctcgcagtgcagatgcccctgcttatcagcagggacagaatcagctgtacaacgagctgaatctgggcaggcgcgaggaatacgacgtgctggataagcgacggggcagagaccccgaaatgggagggaagcccagaaggaaaaaccctcaggaggggctgtataatgaactgcagaaggacaaaatggcagaggcctacagtgaaatcgggatgaagggagagcgccgacggggaaaaggccacgatggactgtatcagggcctgtctactgccaccaaggacacctacgatgccctgcacatgcaggctctgcctccacgctga

SEQ ID NO: 19 - protein, 456 aa, Artificial Sequence
  Description of Artificial Sequence Synthetic polypeptide
MLLLVTSLLLCELPHPAFLLIPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSDKTHTCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - protein, 497 aa, Artificial Sequence
  Description of Artificial Sequence Synthetic polypeptide
MLLLVTSLLLCELPHPAFLLIPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSDKTHTCPPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - protein, 457 aa, Artificial Sequence
  Description of Artificial Sequence Synthetic polypeptide
MLLLVTSLLLCELPHPAFLLIPQVQLVQSGGGVVQPGRSLRLSCAASGFTFSSYGLHWVRQAPGKGLEWVAAISYDGSKKYYADSVKGRLTISRDNSKNTLYLQMNSLRPDDTALYFCARGWFVEPLSWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 1371 bp, Artificial Sequence
  Description of Artificial Sequence Synthetic polynucleotide
atgctgctgctggtgacaagcctgctgctgtgcgaactgccccatcccgccttcctgctgattcctgatgtcgtgatgacgcagagccctctctctcttcccgttacccctggtgaacccgcatcaataagttgccgctccagtcaatcacttgtacattcaaatcgcaatacctacctgcactggtatttgcagaagccgggacaatcccctcaattgttgatatataaggtatccaatcgcttttctggagttcctgatagattcagcggatccgggtctggtactgatttcactctgaaaatatccagggtcgaagctgaggacgtaggcgtatattattgctctcagaacacgcatgtcccgccgactttcggccagggcactaaacttgagatcaagggtggggggggcagcggtggtggaggctctggtggaggagggagccaggtccaactcgttcaaagtggcgcagaggtcaaaaagccaggcgcgagcgttaaagtatcatgtaaggccagcggttatactttcactgattatgaaatgcactgggtgcgacaagcccccgggcaaggtcttgagtggatgggtgcacttgatccaaaaactggggatactgcctatagccagaaattcaaagggcgcgtcacactcactgccgacaaaagtacgagcacagcttatatggaattgagttcactgacgagcgaggatacggcagtttattactgtacgcgcttctactcttacacttattgggggcaaggcactttggttactgtgtcctctgacaagacccatacgtgtccaccgtgtcccttctgggtattggttgtggtcggcggtgtccttgcttgttacagccttctcgtgacagtcgcattcataattttttgggtgaaaagaggtcggaaaaagttgctgtatattttcaaacaaccctttatgagacctgtacaaacgactcaggaagaggatggttgtagttgcaggtttccggaggaggaggaaggtgggtgcgaactgcgggtgaaatttagtagaagcgctgacgcaccagcttaccaacaaggacagaaccaattgtacaacgagcttaacttgggtaggagggaggaatatgatgtactggacaaaaggcgaggtcgcgatccggaaatgggaggcaagccacagcgccggaaaaacccgcaggaaggcttgtacaacgaacttcagaaagataaaatggcagaagcatactccgaaatagggatgaaaggtgaacggcggcgaggcaagggccacgacggtctgtaccaagggttgtcaacggcaactaaagacacgtatgatgcacttcatatgcaagctctgccacccaggtga

SEQ ID NO: 23 - DNA, 1494 bp, Artificial Sequence
  Description of Artificial Sequence Synthetic polynucleotide
atgctgctgctggtgacaagcctgctgctgtgcgaactgccccatcccgccttcctgctgattcctgatgtcgtgatgacgcagagccctctctctcttcccgttacccctggtgaacccgcatcaataagttgccgctccagtcaatcacttgtacattcaaatcgcaatacctacctgcactggtatttgcagaagccgggacaatcccctcaattgttgatatataaggtatccaatcgcttttctggagttcctgatagattcagcggatccgggtctggtactgatttcactctgaaaatatccagggtcgaagctgaggacgtaggcgtatattattgctctcagaacacgcatgtcccgccgactttcggccagggcactaaacttgagatcaagggtggggggggcagcggtggtggaggctctggtggaggagggagccaggtccaactcgttcaaagtggcgcagaggtcaaaaagccaggcgcgagcgttaaagtatcatgtaaggccagcggttatactttcactgattatgaaatgcactgggtgcgacaagcccccgggcaaggtcttgagtggatgggtgcacttgatccaaaaactggggatactgcctatagccagaaattcaaagggcgcgtcacactcactgccgacaaaagtacgagcacagcttatatggaattgagttcactgacgagcgaggatacggcagtttattactgtacgcgcttctactcttacacttattgggggcaaggcactttggttactgtgtcctctgacaagacccatacgtgtccaccgtgtcccttctgggtattggttgtggtcggcggtgtccttgcttgttacagccttctcgtgacagtcgcattcataattttttgggtgcggagcaagcggagccggctgctgcactctgactacatgaacatgactccacggagacccggccctacccggaaacattatcagccctacgccccacccagagattttgccgcttataggtccaaaagaggtcggaaaaagttgctgtatattttcaaacaaccctttatgagacctgtacaaacgactcaggaagaggatggttgtagttgcaggtttccggaggaggaggaaggtgggtgcgaactgcgggtgaaatttagtagaagcgctgacgcaccagcttaccaacaaggacagaaccaattgtacaacgagcttaacttgggtaggagggaggaatatgatgtactggacaaaaggcgaggtcgcgatccggaaatgggaggcaagccacagcgccggaaaaacccgcaggaaggcttgtacaacgaacttcagaaagataaaatggcagaagcatactccgaaatagggatgaaaggtgaacggcggcgaggcaagggccacgacggtctgtaccaagggttgtcaacggcaactaaagacacgtatgatgcacttcatatgcaagctctgccacccaggtga

SEQ ID NO: 24 - DNA, 1374 bp, Artificial Sequence
  Description of Artificial Sequence Synthetic polynucleotide
atgctgctgctggtgacaagcctgctgctgtgcgaactgccccatcccgccttcctgctgattcctcaggtccagcttgtgcaaagcggaggaggagtggtacagcctggccgctctttgagactgtcttgtgcggccagtggatttacattctcttcttatgggttgcattgggtcagacaagcaccgggcaaaggattggaatgggtcgcggccattagctatgatggctcaaagaaatattatgccgattccgtaaaagggaggttgacaataagccgggataacagcaagaacactttgtatcttcagatgaatagcctccgaccggacgacacggcactgtatttttgcgcacgcgggtggtttgtagaacccctgagttggggacaaggtactcttgtcacggtatcttctggcggaggtgggagtggtgggggtggcagtggcgggggtgggtcacaaagcgtgcttacacaacctccttctgcgagcggaactccgggacaacgggttacgatttcatgctccggctcaagtagcaatataggatcaaatacagtgaattggtatcaacaactccctggcacagcgcccaagctgctgatctactctaataaccagaggccgagtggtgtgccagataggttcagtggctctaaatcaggtactagcgcgagcctcgccatttcaggacttcaatcagaggatgaagcggactactactgtgccgcgtgggatgattcacttaatggatatgttttcgggaccggaacaaaattgacggtattggagagcaaatatggaccaccatgccctccatgtcctttttgggtcctggtggtcgtgggaggcgtgctggcatgttattctctgctggtcacagtggctttcatcatcttctgggtcaagcgaggccggaagaaactgctgtacatcttcaaacagccttttatgcgcccagtgcagacaactcaggaggaagacggctgctcttgtcggttccccgaggaagaggaagggggatgtgagctgcgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccaggtga

SEQ ID NO: 25 - protein, 456 aa, Artificial Sequence
  Description of Artificial Sequence Synthetic polypeptide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - DNA, 1368 bp, Artificial Sequence
  Description of Artificial Sequence Synthetic polynucleotide
atgctgctgctggtgacaagcctgctgctgtgcgaactgccccatcccgccttcctgctgattcctgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattctctgctggtcacagtggctttcatcatcttctgggtcaagcgaggccggaagaaactgctgtacatcttcaaacagccttttatgcgcccagtgcagacaactcaggaggaagacggctgctcttgtcggttccccgaggaagaggaagggggatgtgagctgcgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccagg

SEQ ID NO: 27 - protein, 116 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSS

SEQ ID NO: 28 - protein, 110 aa, Artificial Sequence
  Description of Artificial Sequence Synthetic polypeptide
SYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVL

SEQ ID NO: 29 - protein, 116 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSS

SEQ ID NO: 30 - protein, 110 aa, Artificial Sequence
  Description of Artificial Sequence Synthetic polypeptide
QSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVL

SEQ ID NO: 31 - protein, 242 aa, Artificial Sequence
  Description of Artificial Sequence Synthetic polypeptide
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 32 - protein, 242 aa, Artificial Sequence
  Description of Artificial Sequence Synthetic polypeptide
QVQLVQSGGGVVQPGRSLRLSCAASGFTFSSYGLHWVRQAPGKGLEWVAAISYDGSKKYYADSVKGRLTISRDNSKNTLYLQMNSLRPDDTALYFCARGWFVEPLSWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVL

SEQ ID NO: 33 - DNA, 723 bp, Artificial Sequence
  Description of Artificial Sequence Synthetic polynucleotide
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtccta

SEQ ID NO: 34 - DNA, 723 bp, Artificial Sequence
  Description of Artificial Sequence Synthetic polynucleotide
gaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctg

SEQ ID NO: 35 - DNA, 726 bp, Artificial Sequence
  Description of Artificial Sequence Synthetic polynucleotide
gatgtcgtgatgacgcagagccctctctctcttcccgttacccctggtgaacccgcatcaataagttgccgctccagtcaatcacttgtacattcaaatcgcaatacctacctgcactggtatttgcagaagccgggacaatcccctcaattgttgatatataaggtatccaatcgcttttctggagttcctgatagattcagcggatccgggtctggtactgatttcactctgaaaatatccagggtcgaagctgaggacgtaggcgtatattattgctctcagaacacgcatgtcccgccgactttcggccagggcactaaacttgagatcaagggtggggggggcagcggtggtggaggctctggtggaggagggagccaggtccaactcgttcaaagtggcgcagaggtcaaaaagccaggcgcgagcgttaaagtatcatgtaaggccagcggttatactttcactgattatgaaatgcactgggtgcgacaagcccccgggcaaggtcttgagtggatgggtgcacttgatccaaaaactggggatactgcctatagccagaaattcaaagggcgcgtcacactcactgccgacaaaagtacgagcacagcttatatggaattgagttcactgacgagcgaggatacggcagtttattactgtacgcgcttctactcttacacttattgggggcaaggcactttggttactgtgtcctct

SEQ ID NO: 36 - DNA, 726 bp, Artificial Sequence
  Description of Artificial Sequence Synthetic polynucleotide
caggtccagcttgtgcaaagcggaggaggagtggtacagcctggccgctctttgagactgtcttgtgcggccagtggatttacattctcttcttatgggttgcattgggtcagacaagcaccgggcaaaggattggaatgggtcgcggccattagctatgatggctcaaagaaatattatgccgattccgtaaaagggaggttgacaataagccgggataacagcaagaacactttgtatcttcagatgaatagcctccgaccggacgacacggcactgtatttttgcgcacgcgggtggtttgtagaacccctgagttggggacaaggtactcttgtcacggtatcttctggcggaggtgggagtggtgggggtggcagtggcgggggtgggtcacaaagcgtgcttacacaacctccttctgcgagcggaactccgggacaacgggttacgatttcatgctccggctcaagtagcaatataggatcaaatacagtgaattggtatcaacaactccctggcacagcgcccaagctgctgatctactctaataaccagaggccgagtggtgtgccagataggttcagtggctctaaatcaggtactagcgcgagcctcgccatttcaggacttcaatcagaggatgaagcggactactactgtgccgcgtgggatgattcacttaatggatatgttttcgggaccggaacaaaattgacggtattg

SEQ ID NO: 37 - protein, 10 aa, Artificial Sequence
  Description of Artificial Sequence Synthetic peptide
GFTFSSYAMS

SEQ ID NO: 38 - protein, 17 aa, Artificial Sequence
  Description of Artificial Sequence Synthetic peptide
AISGSGGSTYYADSVKG

SEQ ID NO: 39 - protein, 7 aa, Artificial Sequence
  Description of Artificial Sequence Synthetic peptide
GKRYFDY

SEQ ID NO: 40 - protein, 13 aa, Artificial Sequence
  Description of Artificial Sequence Synthetic peptide
SGGSSNIGSNTVN

SEQ ID NO: 41 - protein, 7 aa, Artificial Sequence
  Description of Artificial Sequence Synthetic peptide
FNNQRPS

SEQ ID NO: 42 - protein, 11 aa, Artificial Sequence
  Description of Artificial Sequence Synthetic peptide
VAWDDSLNAPV

SEQ ID NO: 43 - protein, 13 aa, Artificial Sequence
  Description of Artificial Sequence Synthetic peptide
SGGSSDIGSNTVN

SEQ ID NO: 44 - protein, 7 aa, Artificial Sequence
  Description of Artificial Sequence Synthetic peptide
YNNQRPS

SEQ ID NO: 45 - protein, 11 aa, Artificial Sequence
  Description of Artificial Sequence Synthetic peptide
ATWDDRMYSPV

SEQ ID NO: 46 - protein, 15 aa, Artificial Sequence
  Description of Artificial Sequence Synthetic peptide
  MISC_FEATURE (1)..(15): This sequence may encompass 1-3 “Gly Gly Gly Gly Ser” repeating units
GGGGSGGGGSGGGGS